(19) United States
(12) Patent Application Publication
(10) Pub. No.: US 2009/0264402 A1
(43) Pub. Date: Oct. 22, 2009

(54) NOVEL DIPHENYLAZETIDINONE SUBSTITUTED BY PIPERAZINE-1-SULFONIC ACID AND HAVING IMPROVED PHARMACOLOGICAL PROPERTIES

(75) Inventors: Gerhard Jaehne, Frankfurt (DE); Wendelin Frick, Hunstetten-Beuerbach (DE); Andreas Lindenschmidt, Sulzbach (DE); Hubert Heuer, Schwabenheim (DE); Hans-Ludwig Schaefer, Hochheim (DE); Werner Kramer, Mainz-Laubernheim (DE); Claus-Dieter Graf, Hattersheim (DE); Wolfgang Schmider, Frankfurt (DE)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: SANOFI-AVENTIS DEUTSCHLAND GMBH, Frankfurt am Main (DE)

(21) Appl. No.: 12/432,997

(22) Filed: Apr. 30, 2009

Related U.S. Application Data
(63) Continuation of application No. PCT/EP2007/009018, filed on Oct. 18, 2007.

(30) Foreign Application Priority Data
Nov. 2, 2006 (DE) 102006051655.9-44

(51) Int. Cl. A61K 31/496; C07D 403/12; A61P 3/06; C07D 413/12

(52) U.S. Cl. 514/210.02; 544/359; 544/388; 544/369

(57) ABSTRACT

The invention therefore relates to the compound of the formula I

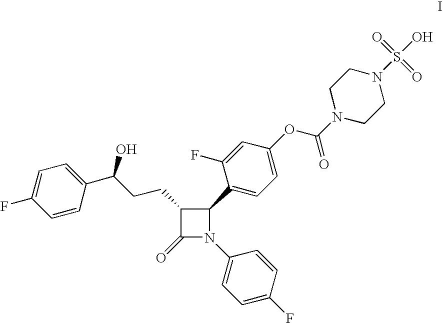

or a pharmaceutically acceptable salt thereof, its pharmaceutically composition and uses.

This application is a Continuation of International Application No. PCT/EP2007/009018, filed Oct. 18, 2007, which is incorporated herein by reference in its entirety.

### FIELD OF THE INVENTION

The invention relates to a diphenylazetidinone substituted by piperazine-1-sulfonic acid and to its physiologically tolerated salts.

### BACKGROUND OF THE INVENTION

Diphenylazetidinones of similar structure and their use for the treatment of hyperlipidemia have already been described (WO 2004/000804).

The invention was based on the object of providing a compound which, in contrast to the compounds described in WO 2004/000804, exhibits a distinctly improved effect. It was particularly intended to provide a diphenylazetidinone substituted by piperazine-1-sulfonic acid and having an improved effect.

### SUMMARY OF THE INVENTION

The invention therefore relates to the compound of the formula I

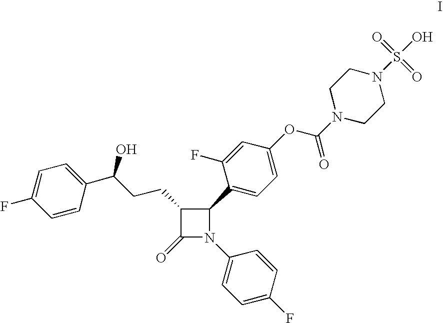

and the pharmaceutically acceptable salts thereof.

### DETAILED DESCRIPTION OF THE INVENTION

Pharmaceutically acceptable salts are, because their solubility in water is greater than that of the initial or basic compounds, particularly suitable for medical applications. These salts must have a pharmaceutically acceptable cation. Suitable pharmaceutically acceptable salts are ammonium salts, alkali metal salts (such as sodium and potassium salts), alkaline earth metal salts (such as magnesium and calcium salts), zinc salts, and salts of trometamol (2-amino-2-hydroxymethyl-1,3-propanediol), diethanolamine, lysine, arginine, choline, meglumine or ethylenediamine salts.

The compound of the invention may also exist in various polymorphous forms, e.g. as amorphous and crystalline polymorphous forms. All polymorphous forms of the compound according to the invention belong within the framework of the invention and are a further aspect of the invention.

All references to “compound(s) of formula I” hereinafter refer to compound(s) of the formula I as described above, and the salts and solvates thereof as described herein.

The compound of the formula I represents an ideal pharmaceutical for the treatment of lipid metabolism disorders, especially hyperlipidemia. The compound of the formula I is likewise suitable for influencing the serum cholesterol level and for the prevention and treatment of arterial sclerotic manifestations.

The compound(s) of the formula (I) can also be administered in combination with further active ingredients.

The amount of a compound of formula I necessary to achieve the desired biological effect depends on a number of factors, for example the specific compound chosen, the intended use, the mode of administration and the clinical condition of the patient. The daily dose is generally in the range from 0.01 mg to 100 mg (typically from 0.05 mg and 50 mg) per day and per kilogram of body weight, for example 0.1-10 mg/kg/day.

Single-dose formulations which can be administered orally, such as, for example, tablets or capsules may contain, for example, from 1.0 to 1000 mg, typically from 10 to 600 mg. For the therapy of the abovementioned conditions, the compound of formula I may be used as the compound itself, but it is preferably in the form of a pharmaceutical composition with an acceptable carrier. The carrier must, of course, be acceptable in the sense that it is compatible with the other ingredients of the composition and is not harmful for the patient's health. The carrier may be a solid or a liquid or both and is preferably formulated with the compound as a single dose, for example as a tablet, which may contain from 0.05% to 95% by weight of the active ingredient. Other pharmaceutically active substances may likewise be present. The pharmaceutical compositions of the invention can be produced by one of the known pharmaceutical methods, which essentially consist of mixing the ingredients with pharmacologically acceptable carriers and/or excipients.

Pharmaceutical compositions of the invention are those suitable for oral and peroral (for example sublingual) administration, although the most suitable mode of administration depends in each individual case on the nature and severity of the condition to be treated and on the nature of the compound of formula I used in each case. Coated formulations and coated slow-release formulations also belong within the framework of the invention. Preference is given to acid- and gastric juice-resistant formulations. Suitable coatings resistant to gastric juice comprise cellulose acetate phthalate, polyvinyl acetate phthalate, hydroxypropylmethylcellulose phthalate and anionic polymers of methacrylic acid and methyl methacrylate.

Suitable pharmaceutical compounds for oral administration may be in the form of separate units such as, for example, capsules, cachets, suckable tablets or tablets, each of which contains a defined amount of the compound of formula I; as powders or granules; as solution or suspension in an aqueous or nonaqueous liquid; or as an oil-in-water or water-in-oil emulsion. These compositions may, as already mentioned, be prepared by any suitable pharmaceutical method which includes a step in which the active ingredient and the carrier (which may consist of one or more additional ingredients) are brought into contact. The compositions are generally produced by uniform and homogeneous mixing of the active ingredient with a liquid and/or finely divided solid carrier, after which the product is shaped if necessary. Thus, for example, a tablet can be produced by compressing or molding a powder or granules of the compound, where appropriate with one or more additional ingredients. Compressed tablets can be produced by tableting the compound in free-flowing form such as, for example, a powder or granules, where appropriate mixed with a binder, glidant, inert diluent and/or one (or more) surface-active/dispersing agent(s) in a suitable machine. Molded tablets can be produced by molding the compound, which is in powder form and is moistened with an inert liquid diluent, in a suitable machine.

Pharmaceutical compositions which are suitable for peroral (sublingual) administration comprise suckable tablets which contain a compound of formula I with a flavoring, normally sucrose and gum arabic or tragacanth, and pastilles which comprise the compound in an inert base such as gelatin and glycerol or sucrose and gum arabic.

Further active ingredients suitable for combination products are:

All antidiabetics which are mentioned in the Rote Liste 2006, chapter 12; all weight-reducing agents/appetite suppressants which are mentioned in the Rote Liste 2006, chapter 1; all lipid-lowering agents which are mentioned in the Rote Liste 2006, chapter 58. They may be combined with the compound of the invention of the formula I in particular for a synergistic improvement in the effect. The active ingredient combination can be administered either by separate administration of the active ingredients to the patient or in the form of combination products in which a plurality of active ingredients is present in a pharmaceutical preparation. Most of the active ingredients mentioned hereinafter are disclosed in the USP Dictionary of USAN and International Drug Names, US Pharmacopeia, Rockville 2001.

Antidiabetics include insulin and insulin derivatives such as, for example, Lantus® (see www.lantus.com) or HMR 1964 or Levemir® (insulin detemir) or those described in WO2005005477 (Novo Nordisk), fast-acting insulins (see U.S. Pat. No. 6,221,633), inhalable insulins such as, for example, Exubera® or oral insulins such as, for example, IN-105 (Nobex) or Oral-Iyn™ (Generex Biotechnology), GLP-1 derivatives and GLP-1 agonists such as, for example, exenatide, liraglutide or those which have been disclosed in WO98/08871 or WO2005027978, WO20066037811, WO2006037810 of Novo Nordisk A/S, in WO01/04156 of Zealand or in WO0/34331 of Beaufour-Ipsen, pramlintide acetate (Symlin; Amylin Pharmaceuticals), BIM-51077, PC-DAC:exendin-4 (an exendin-4 analog covalently bonded to recombinant human albumin), agonists like those described for example in D. Chan et al., Proc. Natl. Acad. Sci. USA 104 (2007) 943, those described in WO2006124529, and orally effective hypoglycemic active ingredients.

Antidiabetics also include agonists of the glucose-dependent insulinotropic polypeptide (GIP) receptor as described for example in WO2006121860.

The orally effective hypoglycemic active ingredients include preferably

sulfonylureas,
biguanidines,
meglitinides,
oxadiazolidinediones,
thiazolidinediones,
glucosidase inhibitors,
inhibitors of glycogen phosphorylase,
glucagon antagonists,
glucokinase activators,
inhibitors of fructose-1,6-bisphosphatase,
modulators of glucose transporter 4 (GLUT4),
inhibitors of glutamine-fructose-6-phosphate amidotransferase (GFAT), GLP-1 agonists,
potassium channel openers such as, for example, pinacidil, cromakalim, diazoxide or those described in R. D. Carr et al., Diabetes 52, 2003, 2513-2518, in J. B. Hansen et al., Current Medicinal Chemistry 11, 2004, 1595-1615, in T. M. Tagmose et al., J. Med. Chem. 47, 2004, 3202-3211 or in M. J. Coghlan et al., J. Med. Chem. 44, 2001, 1627-1653, or those which have been disclosed in WO 97/26265 and WO 99/03861 of Novo Nordisk A/S,
inhibitors of dipeptidylpeptidase IV (DPP-IV),
insulin sensitizers,
inhibitors of liver enzymes involved in stimulating gluconeogenesis and/or glycogenolysis,
modulators of glucose uptake, of glucose transport and of glucose reabsorption,
inhibitors of 11β-HSD1,
inhibitors of protein tyrosine phosphatase 1B (PTP1B),
modulators of the sodium-dependent glucose transporter 1 or 2 (SGLT1, SGLT2),
compounds which alter lipid metabolism such as antihyperlipidemic active ingredients and antilipidemic active ingredients,
compounds which reduce food intake,
compounds which increase thermogenesis,
PPAR and RXR modulators and
active ingredients which act on the ATP-dependent potassium channel of the beta cells.

In one embodiment of the invention, the compound of the formula I is administered in combination with an HMGCOA reductase inhibitor such as simvastatin, fluvastatin, pravastatin, lovastatin, atorvastatin, cerivastatin, rosuvastatin, L-659699.

In one embodiment of the invention, the compound of the formula I is administered in combination with a cholesterol absorption inhibitor such as, for example, ezetimibe, tiqueside, pamaqueside, FM-VP4 (sitostanol/campesterol ascorbyl phosphate; Forbes Medi-Tech, WO2005042692, WO2005005453), MD-0727 (Microbia Inc., WO2005021497, WO2005021495) or with compounds as described in WO2002066464, WO2005000353 (Kotobuki Pharmaceutical Co. Ltd.), or WO2005044256 or WO2005062824 (Merck & Co.) or WO2005061451 and WO2005061452 (AstraZeneca AB), and WO2006017257 (Phenomix) or WO2005033100 (Lipideon Biotechnology AG) or as described in WO2004097655, WO2004000805, WO2004000804, WO2004000803, WO2002050068, WO2002050060, WO2005047248, WO2006086562, WO2006102674, WO2006116499, WO2006121861, WO2006122186, WO2006122216, WO2006127893, WO2006137794, WO2006137796, WO2006137782, WO2006137793, WO2006137797, WO2006137795, WO2006137792, WO2006138163.

In one embodiment of the invention, the compound of the formula I is administered in combination with Vytorin™, a fixed combination of ezetimibe with simvastatin.

In one embodiment of the invention, the compound of the formula I is administered in combination with a fixed combination of ezetimibe with atorvastatin.

In one embodiment of the invention, the compound of the formula I is administered in combination with a fixed combination of ezetimibe with fenofibrate.

In a further embodiment of the invention, the compound of the formula I is administered in combination with a fixed combination of fenofibrate with rosuvastatin.

In a further embodiment of the invention, the compound of the formula I is administered in combination with synordia (R), a fixed combination of fenofibrate with metformin.

In one embodiment of the invention, the compound of the formula I is administered in combination with ISIS-301012, an antisense oligonucleotide able to regulate the apolipoprotein B gene.

In one embodiment of the invention, the compound of the formula I is administered in combination with a PPAR gamma agonist such as, for example, rosiglitazone, pioglitazone, JTT-501, GI 262570, R-483 or CS-011 (rivoglitazone).

In one embodiment of the invention, the compound of the formula I is administered in combination with Competact™, a fixed combination of pioglitazone hydrochloride with metformin hydrochloride.

In one embodiment of the invention, the compound of the formula I is administered in combination with Tandemact™, a fixed combination of pioglitazone with glimepride.

In a further embodiment of the invention, the compound of the formula I is administered in combination with a fixed combination of pioglitazone hydrochloride with an angiotensin II antagonist such as, for example, TAK-536.

In one embodiment of the invention, the compound of the formula I is administered in combination with a PPAR alpha agonist such as, for example, GW9578, GW-590735, K-111, LY-674, KRP-101, DRF-10945, LY-518674 or those described in WO2001040207, WO2002096894, WO2005097076.

In one embodiment of the invention, the compound of the formula I is administered in combination with a mixed PPAR alpha/gamma agonist such as, for example, naveglitazar, LY-510929, ONO-5129, E-3030, AVE 8042, AVE 8134, AVE 0847, CKD-501 (lobeglitazone sulfate) or as described in PCT/US00/11833, PCT/US00/11490, DE10142734.4 or in J. P. Berger et al., TRENDS in Pharmacological Sciences 28(5), 244-251, 2005.

In one embodiment of the invention, the compound of the formula I is administered in combination with a PPAR delta agonist such as, for example, GW-501516 or as described in WO2006059744, WO2006084176, WO2006029699, WO2007039172, WO2007039178.

In one embodiment of the invention, the compound of the formula I is administered in combination with metaglidasen or with MBX-2044 or other partial PPAR gamma agonists/antagonists.

In one embodiment of the invention, the compound of the formula I is administered in combination with a fibrate such as, for example, fenofibrate, clofibrate or bezafibrate.

In one embodiment of the invention, the compound of the formula I is administered in combination with an MTP inhibitor such as, for example, implitapide, BMS-201038, R-103757, AS-1552133 or those described in WO2005085226, WO2005121091, WO2006010423.

In one embodiment of the invention, the compound of the formula I is administered in combination with a CETP inhibitor such as, for example, torcetrapib or JTT-705 or those described in WO2006002342, WO2006010422, WO2006012093, WO2006073973, WO2006072362, WO2006097169, WO2007041494.

In one embodiment of the invention, the compound of the formula I is administered in combination with a bile acid absorption inhibitor (see, for example, U.S. Pat. No. 6,245,744, U.S. Pat. No. 6,221,897 or WO0/61568), such as, for example, HMR 1741 or those as described in DE 10 2005 033099.1 and DE 10 2005 033100.9, WO2007009655-56.

In one embodiment of the invention, the compound of the formula I is administered in combination with a polymeric bile acid adsorbent such as, for example, cholestyramine or colesevelam.

In one embodiment of the invention, the compound of the formula I is administered in combination with an LDL receptor inducer (see U.S. Pat. No. 6,342,512), such as, for example, HMR1171, HMR1586 or those as described in WO2005097738.

In one embodiment of the invention, the compound of the formula I is administered in combination with an ABCA1 expression enhancer as described for example in WO2006072393.

In a further embodiment of the invention, the compound of the formula I is administered in combination with an RNAi therapeutic directed against PCSK9 (proprotein convertase subtilisin/kexin type 9).

In one embodiment, the compound of the formula I is administered in combination with Omacor® (omega-3 fatty acids; highly concentrated ethyl esters of eicosapentaenoic acid and of docosahexaenoic acid).

In one embodiment of the invention, the compound of the formula I is administered in combination with an ACAT inhibitor such as, for example, avasimibe or SMP-797.

In one embodiment of the invention, the compound of the formula I is administered in combination with an antioxidant such as, for example, OPC-14117, probucol, tocopherol, ascorbic acid, β-carotene or selenium.

In one embodiment of the invention, the compound of the formula I is administered in combination with a vitamin such as, for example, vitamin B6 or vitamin B12.

In one embodiment of the invention, the compound of the formula I is administered in combination with a lipoprotein lipase modulator such as, for example, ibrolipim (NO-1886).

In one embodiment of the invention, the compound of the formula I is administered in combination with an ATP citrate lyase inhibitor such as, for example, SB-204990.

In one embodiment of the invention, the compound of the formula I is administered in combination with a squalene synthetase inhibitor such as, for example, BMS-188494, TAK-475 or as described in WO2005077907, JP2007022943.

In one embodiment of the invention, the compound of the formula I is administered in combination with a lipoprotein (a) antagonist such as, for example, gemcabene (CI-1027).

In one embodiment of the invention, the compound of the formula I is administered in combination with an agonist of GPR109A (HM74A receptor agonist; NAR agonist (nicotinic acid receptor agonist)) such as, for example, nicotinic acid or extended release niacin in conjunction with MK-0524A or the compounds described in WO2006045565, WO2006045564, WO2006069242, WO2006124490, WO2006113150, WO2007017261, WO2007017262, WO2007017265, WO2007015744, WO2007027532.

In another embodiment of the invention, the compound of the formula I is administered in combination with an agonist of GPR116 as described for example in WO2006067531, WO2006067532.

In one embodiment of the invention, the compound of the formula I is administered in combination with a lipase inhibitor such as, for example, orlistat or cetilistat (ATL-962).

In one embodiment of the invention, the compound of the formula I is administered in combination with insulin.

In one embodiment of the invention, the compound of the formula I is administered in combination with a sulfonylurea such as, for example, tolbutamide, glibenclamide, glipizide or glimepiride.

In one embodiment, the compound of the formula I is administered in combination with a substance which enhances insulin secretion, such as, for example, KCP-265 (WO2003097064) or those described in WO2007026761.

In one embodiment, the compound of the formula I is administered in combination with agonists of the glucose-dependent insulinotropic receptor (GDIR), such as, for example, APD-668.

In one embodiment of the invention, the compound of the formula I is administered in combination with a biguanide such as, for example, metformin.

In another embodiment of the invention, the compound of the formula I is administered in combination with a meglitinide such as, for example, repaglinide, nateglinide or mitiglinide.

In a further embodiment, the compound of the formula I is administered with a combination of mitiglinide with a glitazone, e.g. pioglitazone hydrochloride.

In a further embodiment, the compound of the formula I is administered with a combination of mitiglinide with an alpha-glucosidase inhibitor.

In one embodiment of the invention, the compound of the formula I is administered in combination with a thiazolidinedione such as, for example, troglitazone, ciglitazone, pioglitazone, rosiglitazone or the compounds disclosed in WO 97/41097 of Dr. Reddy's Research Foundation, in particular 5-[[4-[(3,4-dihydro-3-methyl-4-oxo-2-quinazolinylmethoxy]phenyl]methyl]-2,4-thiazolidinedione.

In one embodiment of the invention, the compound of the formula I is administered in combination with an α-glucosidase inhibitor such as, for example, miglitol or acarbose.

In one embodiment of the invention, the compound of the formula I is administered in combination with an active ingredient which acts on the ATP-dependent potassium channel of the beta cells, such as, for example, tolbutamide, glibenclamide, glipizide, glimepiride or repaglinide.

In one embodiment of the invention, the compound of the formula I is administered in combination with more than one of the aforementioned compounds, e.g. in combination with a sulfonylurea and metformin, a sulfonylurea and acarbose, repaglinide and metformin, insulin and a sulfonylurea, insulin and metformin, insulin and troglitazone, insulin and lovastatin, etc.

In one embodiment of the invention, the compound of the formula I is administered in combination with an inhibitor of glycogen phosphorylase, such as, for example, PSN-357 or FR-258900 or those as described in WO2003084922, WO2004007455, WO2005073229-31 or WO2005067932.

In one embodiment of the invention, the compound of the formula I is administered in combination with glucagon receptor antagonists such as, for example, A-770077, NNC-25-2504 or as described in WO2004100875 or WO2005065680.

In one embodiment of the invention, the compound of the formula I is administered in combination with activators of glucokinase, such as, for example, LY-2121260 (WO2004063179), PSN-105, PSN-110, GKA-50 or those as are described for example in WO2004072031, WO2004072066, WO2005080360, WO2005044801, WO2006016194, WO2006058923, WO2006112549, WO2006125972, WO2007017549, WO2007017649, WO2007007910, WO2007007040-42, WO2007006760-61, WO2007006814, WO2007007886, WO2007028135, WO2007031739, WO2007041365, WO2007041366, WO2007037534, WO2007043638, WO2007053345, WO2007051846, WO2007051845, WO2007053765, WO2007051847.

In one embodiment of the invention, the compound of the formula I is administered in combination with an inhibitor of gluconeogenesis, such as, for example, FR-225654.

In one embodiment of the invention, the compound of the formula I is administered in combination with inhibitors of fructose-1,6-bisphosphatase (FBPase), such as, for example, CS-917 (MB-06322) or MB-07803 or those described in WO2006023515, WO2006104030, WO2007014619.

In one embodiment of the invention, the compound of the formula I is administered in combination with modulators of glucose transporter 4 (GLUT4), such as, for example, KST-48 (D.-O. Lee et al.: Arzneim.-Forsch. Drug Res. 54 (12), 835 (2004)).

In one embodiment of the invention, the compound of the formula I is administered in combination with inhibitors of glutamine-fructose-6-phosphate amidotransferase (GFAT), as are described for example in WO2004101528.

In one embodiment of the invention, the compound of the formula I is administered in combination with inhibitors of dipeptidylpeptidase IV (DPP-IV), such as, for example, vildagliptin (LAF-237), sitagliptin (MK-0431), sitagliptin phosphate, saxagliptin (BMS-477118), GSK-823093, PSN-9301, SYR-322, SYR-619, TA-6666, TS-021, GRC-8200, GW-825964X, KRP-104, DP-893, ABT-341, ABT-279 or another salt thereof, or the compounds described in WO2003074500, WO2003106456, WO2004037169, WO200450658, WO2005058901, WO2005012312, WO2005/012308, WO2006039325, WO2006058064, PCT/EP2005/007821, PCT/EP2005/008005, PCT/EP2005/008002, PCT/EP2005/008004, PCT/EP2005/008283, DE 10 2005 012874.2, DE 10 2005 012873.4, JP2006160733, WO2006071752, WO2006065826, WO2006078676, WO2006073167, WO2006068163, WO2006090915, WO2006104356, WO2006127530, WO2006111261, WO2007015767, WO2007024993, WO2007029086.

In one embodiment, the compound of the formula I is administered in combination with Janumet™, a fixed combination of sitagliptin phosphate with metformin hydrochloride.

In one embodiment, the compound of the formula I is administered in combination with inhibitors of 11-beta-hydroxysteroid dehydrogenase 1 (11β-HSD1), such as, for example, BVT-2733, JNJ-25918646, INCB-13739 or those as are described for example in WO200190090-94, WO200343999, WO2004112782, WO200344000, WO200344009, WO2004112779, WO2004113310, WO2004103980, WO2004112784, WO2003065983, WO2003104207, WO2003104208, WO2004106294, WO2004011410, WO2004033427, WO2004041264, WO2004037251, WO2004056744, WO2004058730, WO2004065351, WO2004089367, WO2004089380, WO2004089470-71, WO2004089896, WO2005016877, WO2005097759, WO2006010546, WO2006012227, WO2006012173, WO2006017542, WO2006034804, WO2006040329, WO2006051662, WO2006048750, WO2006049952, WO2006048331, WO2006050908, WO2006024627, WO2006040329, WO2006066109, WO2006074244, WO2006078006, WO2006106423, WO2006132436, WO2006134481, WO2006134467, WO2006135795, WO2006136502, WO2006138695, WO2006133926, WO2007003521, WO2007007688, US2007066584, WO2007047625, WO2007051811, WO2007051810.

In one embodiment, the compound of the formula I is administered in combination with inhibitors of protein tyrosine phosphatase 1B (PTP1B), as are described for example in WO200119830-31, WO200117516, WO2004506446, WO2005012295, WO2005116003, PCT/EP2005/005311, PCT/EP2005/005321, PCT/EP2005/007151, DE 10 2004 060542.4, WO2007009911, WO2007028145.

In one embodiment, the compound of the formula I is administered in combination with modulators of the sodium-dependent glucose transporter 1 or 2 (SGLT1, SGLT2), such as, for example, KGA-2727, T-1095, SGL-0010, AVE 2268, SAR 7226 and sergliflozin or as are described for example in WO2004007517, WO200452903, WO200452902, PCT/EP2005/005959, WO2005085237, JP2004359630, WO2005121161, WO2006018150, WO2006035796, WO2006062224, WO2006058597, WO2006073197, WO2006080577, WO2006087997, WO2006108842, WO2007000445, WO2007014895 or by A. L. Handlon in Expert Opin. Ther. Patents (2005) 15(11), 1531-1540.

In one embodiment, the compound of the formula I is administered in combination with modulators of GPR40 as described for example in WO2007013689, WO2007033002.

In one embodiment, the compound of the formula I is administered in combination with modulators of GPR119b as described for example in WO2004041274.

In one embodiment, the compound of the formula I is administered in combination with modulators of GPR119 as described for example in WO2005061489 (PSN-632408), WO2004065380, WO2007003960-62 and WO2007003964.

In a further embodiment, the compound of the formula I is administered in combination with modulators of GPR120.

In one embodiment, the compound of the formula I is administered in combination with inhibitors of hormone-sensitive lipase (HSL) and/or phospholipases as described for example in WO2005073199, WO2006074957, WO2006087309, WO2006111321, WO2007042178.

In one embodiment, the compound of the formula I is administered in combination with inhibitors of acetyl-CoA carboxylase (ACC), such as, for example, those as described in WO199946262, WO200372197, WO2003072197, WO2005044814, WO2005108370, JP2006131559, WO2007011809, WO2007011811, WO2007013691.

In a further embodiment, the compound of the formula I is administered in combination with modulators of xanthine oxidoreductase (XOR).

In one embodiment of the invention, the compound of the formula I is administered in combination with an inhibitor of phosphoenolpyruvate carboxykinase (PEPCK), such as, for example, those as described in WO2004074288.

In one embodiment of the invention, the compound of the formula I is administered in combination with an inhibitor of glycogen synthase kinase 3 beta (GSK-3 beta), as described for example in US2005222220, WO2005085230, PCT/EP2005/005346, WO2003078403, WO2004022544, WO2003106410, WO2005058908, US2005038023, WO2005009997, US2005026984, WO2005000836, WO2004106343, EP1460075, WO2004014910, WO2003076442, WO2005087727 or WO2004046117.

In one embodiment, the compound of the formula I is administered in combination with an inhibitor of the serum/glucocorticoid-regulated kinase (SGK) as described for example in WO2006072354.

In one embodiment, the compound of the formula I is administered in combination with an agonist of the RUP3 receptor as described for example in WO2007035355.

In one embodiment, the compound of the formula I is administered in combination with an inhibitor of protein kinase C beta (PKC beta), such as, for example, ruboxistaurin.

In another embodiment, the compound of the formula I is administered in combination with an activator of the gene which codes for the ataxia telangiectasia mutated (ATM) protein kinase, such as, for example, chloroquine.

In one embodiment, the compound of the formula I is administered in combination with an endothelin A receptor antagonist such as, for example, avosentan (SPP-301).

In one embodiment, the compound of the formula I is administered in combination with inhibitors of “I-kappaB kinase” (IKK inhibitors), as are described for example in WO2001000610, WO2001030774, WO2004022553 or WO2005097129.

In one embodiment, the compound of the formula I is administered in combination with modulators of the glucocorticoid receptor, like those described for example in WO2005090336, WO2006071609, WO2006135826.

In a further embodiment, the compound of the formula I is administered in combination with CART modulators (see “Cocaine-amphetamine-regulated transcript influences energy metabolism, anxiety and gastric emptying in mice” Asakawa, A. et al.: Hormone and Metabolic Research (2001), 33(9), 554-558);

NPY antagonists such as, for example, naphthalene-1-sulfonic acid {4-[(4-aminoquinazolin-2-ylamino)methyl]cyclohexylmethyl}amide hydrochloride (CGP 71683A);
NPY-5 receptor antagonists such as L-152804 or such as, for example as in WO2006001318;
NPY-4 receptor antagonists such as, for example in WO2007038942;
NPY-2 receptor antagonists such as, for example in WO2007038943;
peptide YY 3-36 (PYY3-36) or analogous compounds, such as, for example, CJC-1682 (PYY3-36 conjugated with human serum albumin via Cys34), CJC-1643 (derivative of PYY3-36 which conjugates in vivo to serum albumin) or those as are described in WO2005080424, WO2006095166;
derivatives of the peptide obestatin such as those described in WO2006096847;
CB1R (cannabinoid receptor 1) antagonists (such as, for example, rimonabant, SR147778, SLV-319, AVE-1625, MK-0364 or salts thereof or compounds such as those described for example in EP 0656354, WO 00/15609, WO2001/64632, WO2001/64633, WO2001/64634, WO02/076949, WO2005080345, WO2005080328, WO2005080343, WO2005075450, WO2005080357, WO200170700, WO2003026647-48, WO200302776, WO2003040107, WO2003007887, WO2003027069, U.S. Pat. No. 6,509,367, WO200132663, WO2003086288, WO2003087037, WO2004048317, WO2004058145, WO2003084930, WO2003084943, WO2004058744, WO2004013120, WO2004029204, WO2004035566, WO2004058249, WO2004058255, WO2004058727, WO2004069838, US20040214837, US20040214855, US20040214856, WO2004096209, WO2004096763, WO2004096794, WO2005000809, WO2004099157, US20040266845, WO2004110453, WO2004108728, WO2004000817, WO2005000820, US20050009870, WO200500974, WO2004111033-34, WO200411038-39, WO2005016286, WO2005007111, WO2005007628, US20050054679, WO2005027837, WO2005028456, WO2005063761-62, WO2005061509, WO2005077897, WO2006047516, WO2006060461, WO2006067428, WO2006067443, WO2006087480, WO2006087476, WO2006100208, WO2006106054, WO2006111849, WO2006113704, WO2007009705, WO2007017124, WO2007017126, WO2007018459, WO2007016460, WO2007020502, WO2007026215, WO2007028849, WO2007031720, WO2007031721, WO2007036945, WO2007038045, WO2007039740, US20070015810, WO2007046548, WO2007047737);
cannabinoid receptor 1/cannabinoid receptor 2 (CB1/CB2) modulating compounds as described for example in WO2007001939, WO2007044215, WO2007047737;
MC4 agonists (e.g. 1-amino-1,2,3,4-tetrahydronaphthalene-2-carboxylic acid [2-(3a-benzyl-2-methyl-3-oxo-2,3,3a,4,6,7-hexahydropyrazolo[4,3-c]pyridin-5-yl)-1-(4-chlorophenyl)-2-oxoethyl]amide; (WO 01/91752)) or LB53280, LB53279, LB53278 or THIQ, MB243, RY764, CHIR-785, PT-141 or those that are described in WO2005060985, WO2005009950, WO2004087159, WO2004078717, WO2004078716, WO2004024720, US20050124652, WO2005051391, WO2004112793, WOUS20050222014, US20050176728, US20050164914, US20050124636, US20050130988, US20040167201, WO2004005324, WO2004037797, WO2005042516, WO2005040109, WO2005030797, US20040224901, WO200501921, WO200509184, WO2005000339, EP1460069, WO2005047253, WO2005047251, WO2005118573, EP1538159, WO2004072076, WO2004072077, WO2006021655-57, WO2007009894, WO2007015162, WO2007041061, WO2007041052;
orexin receptor antagonists (e.g. 1-(2-methylbenzoxazol-6-yl)-3-[1,5]naphthyridin-4-ylurea hydrochloride (SB-334867-A) or those as are described for example in WO200196302, WO200185693, WO2004085403, WO2005075458, WO200667224);
histamine H3 receptor agonists (e.g. 3-cyclohexyl-1-(4,4-dimethyl-1,4,6,7-tetrahydroimidazo[4,5-c]pyridin-5-yl)propan-1-one oxalic acid salt (WO 00/63208) or those as are described in WO200064884, WO2005082893, WO2006107661, WO2007003804, WO2007016496, WO2007020213);
histamine H1/histamine H3 modulators such as, for example, betahistine or its dihydrochloride;
CRF antagonists (e.g. [2-methyl-9-(2,4,6-trimethylphenyl)-9H-1,3,9-triazafluoren-4-yl]dipropylamine (WO 00/66585));
CRF BP antagonists (e.g. urocortin);
urocortin agonists;
agonists of the beta-3 adrenoreceptor such as, for example, 1-(4-chloro-3-methanesulfonylmethylphenyl)-2-[2-(2,3-dimethyl-1H-indol-6-yloxy)ethylamino]ethanol hydrochloride (WO 01/83451) or solabegron (GW-427353) or N-5984 (KRP-204), or those as are described in JP2006111553, WO2002038543, WO2007048840-843;
MSH (melanocyte-stimulating hormone) agonists;
MCH (melanin-concentrating hormone) receptor antagonists (such as, for example, NBI-845, A-761, A-665798, A-798, ATC-0175, T-226296, T-71, GW-803430 or compounds such as are described in WO2005085200, WO2005019240, WO2004011438, WO2004012648, WO2003015769, WO2004072025, WO2005070898, WO2005070925, WO2004039780, WO2004092181, WO2003033476, WO2002006245, WO2002089729, WO2002002744, WO2003004027, FR2868780, WO2006010446, WO2006038680, WO2006044293, WO2006044174, JP2006176443, WO2006018280, WO2006018279, WO2006118320, WO2006130075, WO2007018248, WO2007012661, WO2007029847, WO2007024004, WO2007039462, WO2007042660, WO2007042668, WO2007042669, US2007093508, US2007093509, WO2007048802, JP2007091649);
CCK-A agonists (such as, for example, {2-[4-(4-chloro-2,5-dimethoxyphenyl)-5-(2-cyclohexylethyl)thiazol-2-ylcarbamoyl]-5,7-dimethylindol-1-yl}acetic acid trifluoroacetic acid salt (WO 99/15525), SR-146131 (WO 0244150) or SSR-125180) or those as are described in WO2005116034;
serotonin reuptake inhibitors (e.g. dexfenfluramine);
mixed serotonin/dopamine reuptake inhibitors (e.g. bupropion) or fixed combinations of bupropion with naltrexone;
mixed serotoninergic and noradrenergic compounds (e.g. WO 00/71549);
5-HT receptor agonists, e.g. 1-(3-ethylbenzofuran-7-yl)piperazine oxalic acid salt (WO 01/09111);
mixed dopamine/norepinephrine/acetylcholine reuptake inhibitors (e.g. tesofensine);
5-HT2C receptor agonists (such as, for example, lorcaserin hydrochloride (APD-356) BVT-933 or those as are described in WO200077010, WO20077001-02, WO2005019180, WO2003064423, WO200242304, WO2005035533, WO2005082859, WO2006077025, WO2006103511);
5-HT6 receptor modulators such as, for example E-6837 or BVT-74316 or those as are described in WO2005058858, WO2007054257;
bombesin receptor agonists (BRS-3 agonists);
galanin receptor antagonists;
growth hormone (e.g. human growth hormone or AOD-9604);
growth hormone-releasing compounds (tertiary butyl 6-benzyloxy-1-(2-diisopropylaminoethylcarbamoyl)-3,4-dihydro-1H-isoquinoline-2-carboxylate (WO 01/85695));
growth hormone secretagogue receptor antagonists (ghrelin antagonists) such as, for example, A-778193 or those as are described in WO2005030734;
TRH agonists (see, for example, EP 0 462 884);
uncoupling protein 2 or 3 modulators;
leptin agonists (see, for example, Lee, Daniel W.; Leinung, Matthew C.; Rozhavskaya-Arena, Marina; Grasso, Patricia. Leptin agonists as a potential approach to the treatment of obesity. Drugs of the Future (2001), 26(9), 873-881);
DA agonists (bromocriptine or Doprexin);
lipase/amylase inhibitors (like those described for example in WO 00/40569);
inhibitors of diacylglycerol O-acyltransferases (DGATs) such as for example BAY-74-4113 as described for example in US2004/0224997, WO2004094618, WO200058491, WO2005044250, WO2005072740, JP2005206492, WO2005013907, WO2006004200, WO2006019020, WO2006064189, WO2006082952, WO2006120125, WO2006113919, WO2006134317, WO2007016538;
inhibitors of fatty acid synthase (FAS) such as, for example, C75 or those as described in WO2004005277;
inhibitors of stearoyl-CoA delta9 desaturase (SCD1) as described for example in WO2007009236, WO2007044085, WO2007046867, WO2007046868, WO20070501124;
oxyntomodulin;
oleoyl-estrone
or thyroid hormone receptor agonists or partial agonists such as, for example: KB-2115 or those as described in WO20058279, WO200172692, WO200194293, WO2003084915, WO2004018421, WO2005092316, WO2007003419, WO2007009913, WO2007039125.

In one embodiment, the further active ingredient is varenicline tartrate, a partial agonist of the alpha 4-beta 2 nicotinic acetylcholine receptor.

In one embodiment, the further active ingredient is trodusquemine.

In one embodiment, the further active ingredient is a modulator of the SIRT1 enzyme.

In one embodiment of the invention, the further active ingredient is leptin; see, for example, “Perspectives in the therapeutic use of leptin”, Salvador, Javier; Gomez-Ambrosi, Javier; Fruhbeck, Gema, Expert Opinion on Pharmacotherapy (2001), 2(10), 1615-1622.

In one embodiment, the further active ingredient is dexamphetamine or amphetamine.

In one embodiment, the further active ingredient is fenfluramine or dexfenfluramine.

In another embodiment, the further active ingredient is sibutramine.

In one embodiment, the further active ingredient is mazindole or phentermine.

In one embodiment, the further active ingredient is a diphenylazetidinone derivative as described for example in U.S. Pat. No. 6,992,067 or U.S. Pat. No. 7,205,290.

In one embodiment, the compound of the formula I is administered in combination with bulking agents, preferably insoluble bulking agents (see, for example, Carob/Caromax® (Zunft H J; et al., Carob pulp preparation for treatment of hypercholesterolemia, ADVANCES IN THERAPY (2001 September-October), 18(5), 230-6). Caromax is a carob-containing product from Nutrinova, Nutrition Specialties & Food Ingredients GmbH, Industriepark Höchst, 65926 Frankfurt/Main)). Combination with Caromax® is possible in one preparation or by separate administration of compounds of the formula I and Caromax®. Caromax® can in this connection also be administered in the form of food products such as, for example, in bakery products or muesli bars.

It will be understood that every suitable combination of the compounds of the invention with one or more of the aforementioned compounds and optionally one or more further pharmacologically active substances will be regarded as falling within the protection conferred by the present invention.

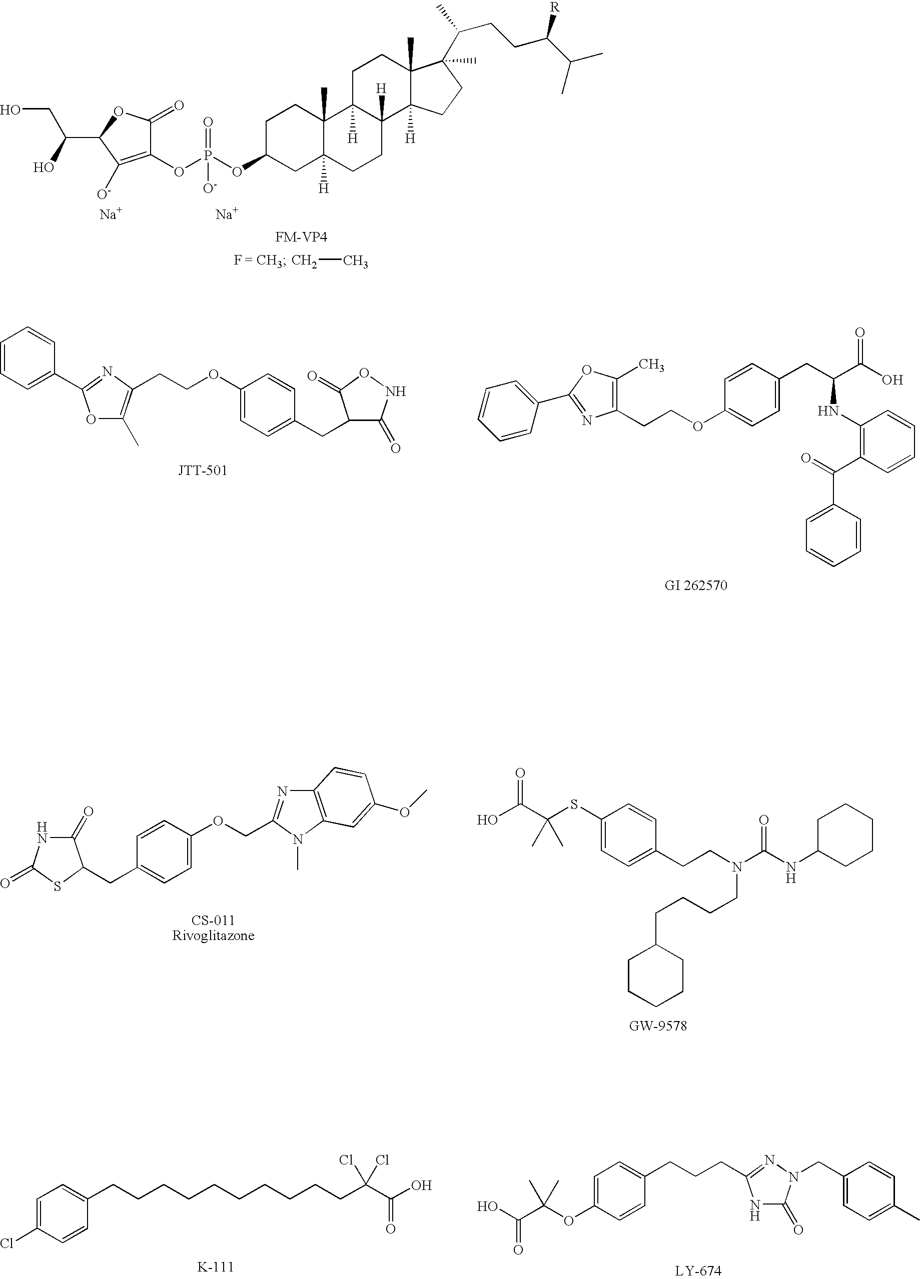

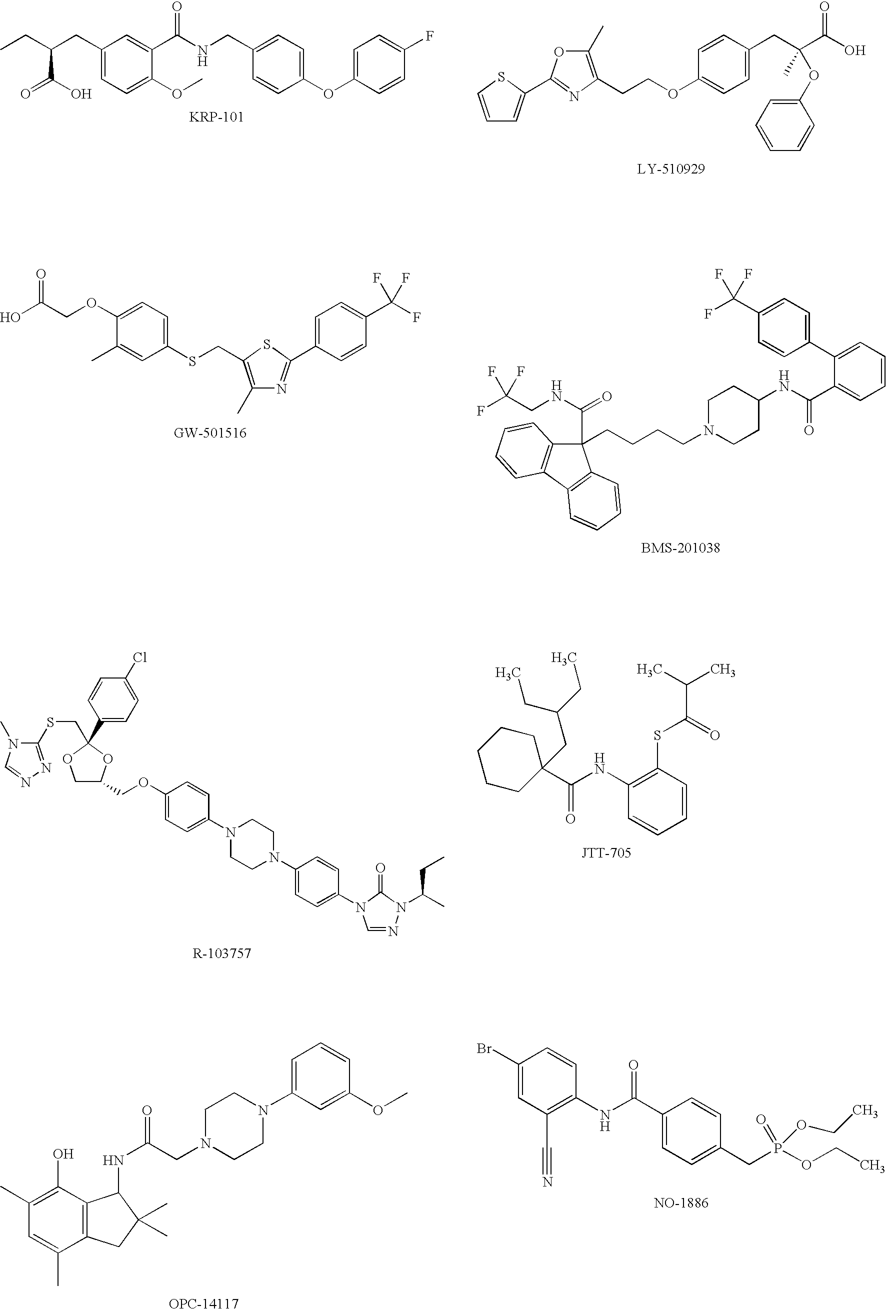

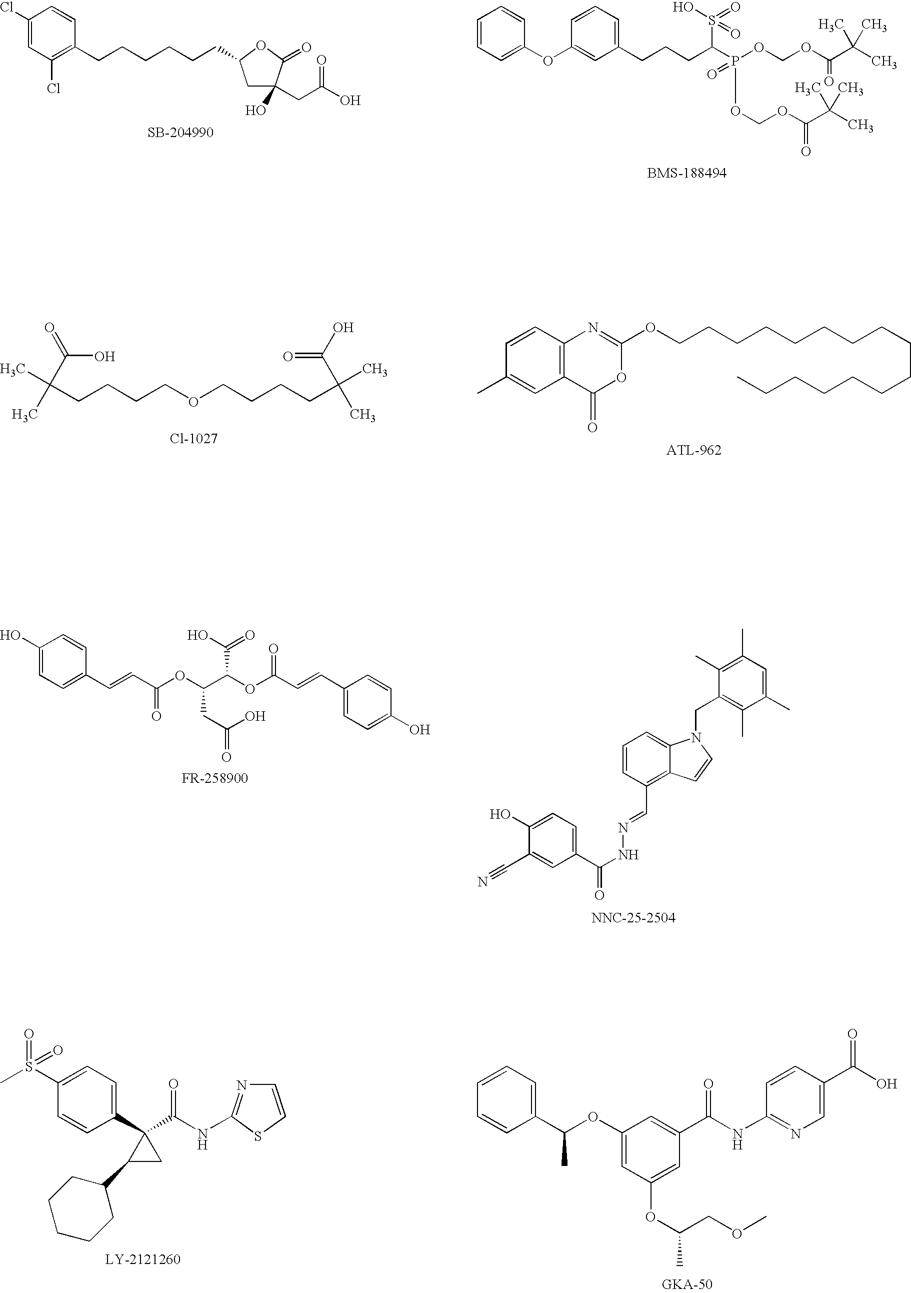

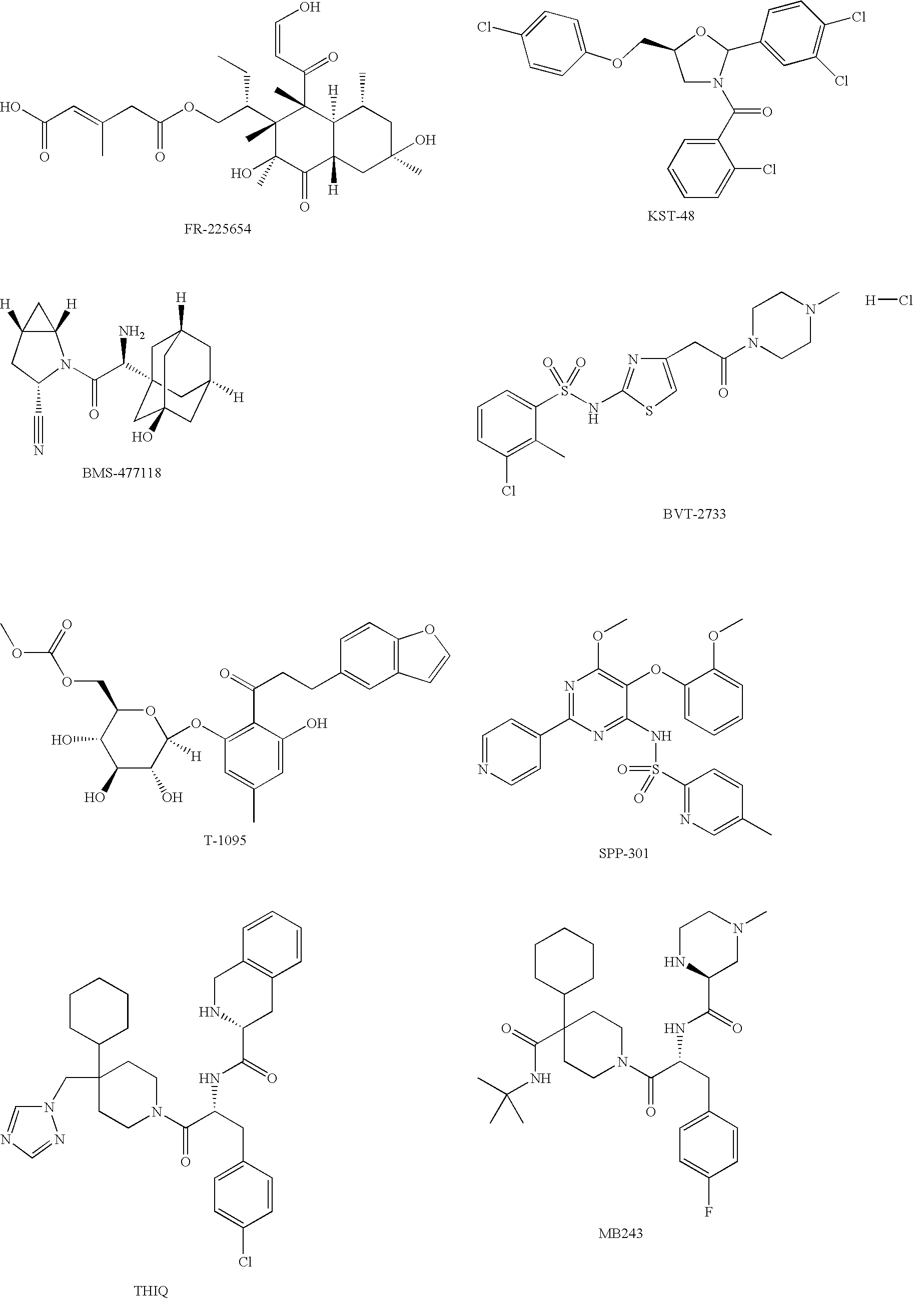

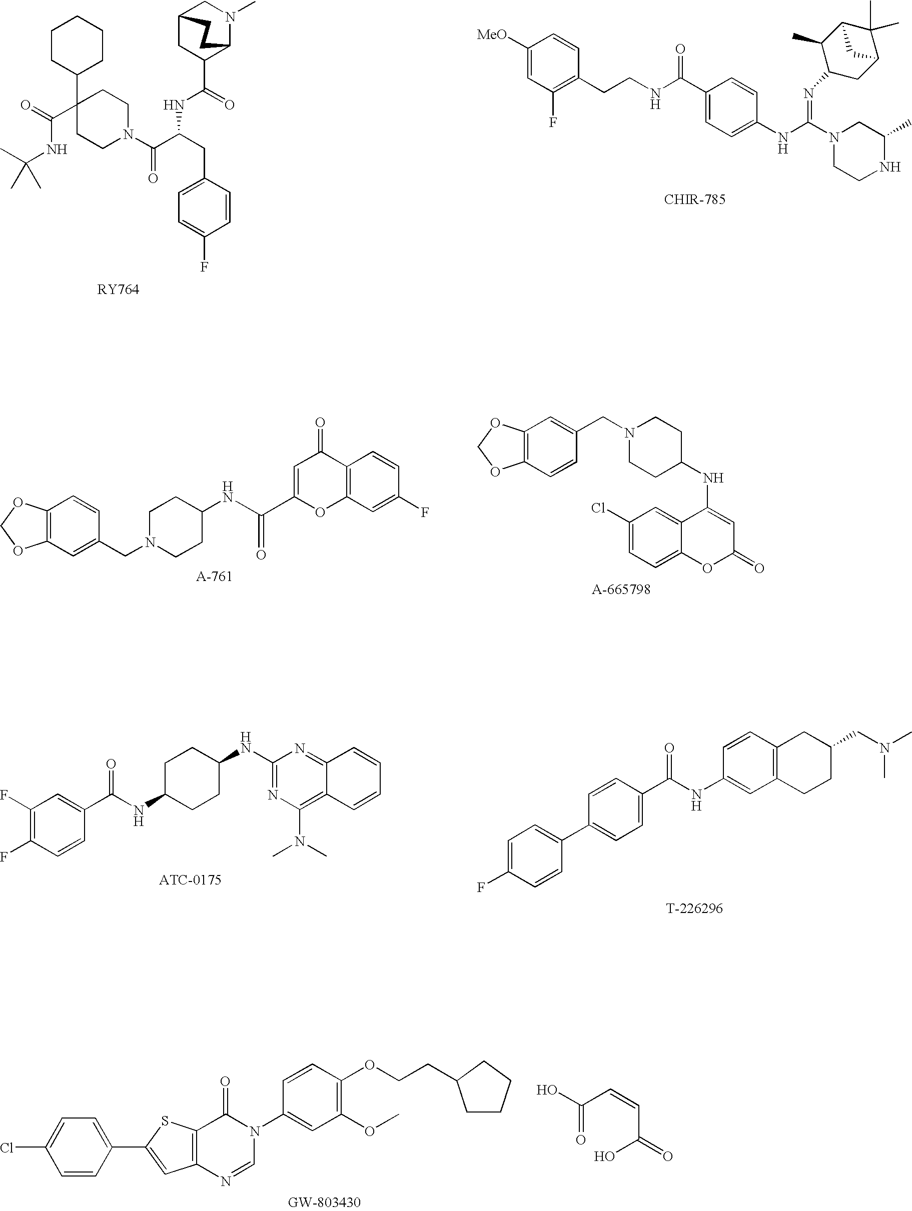

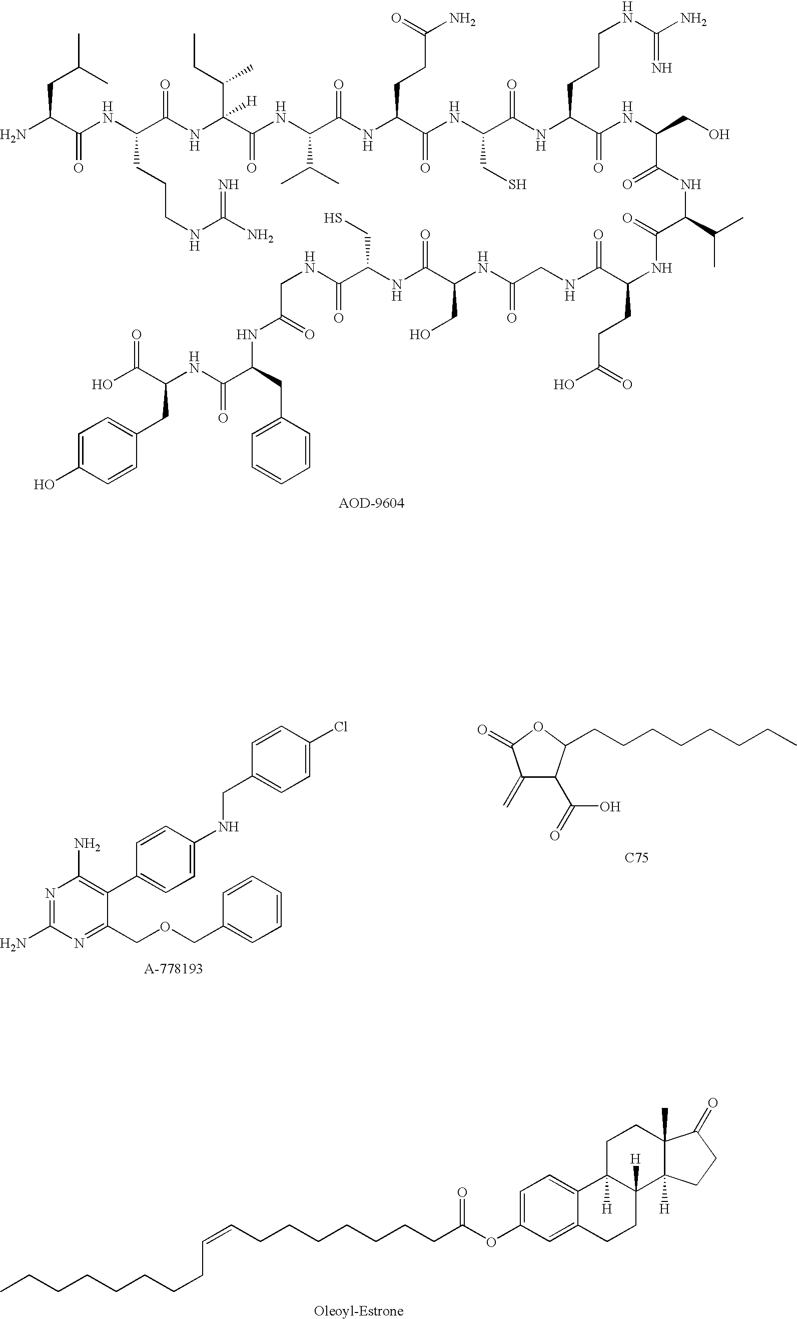

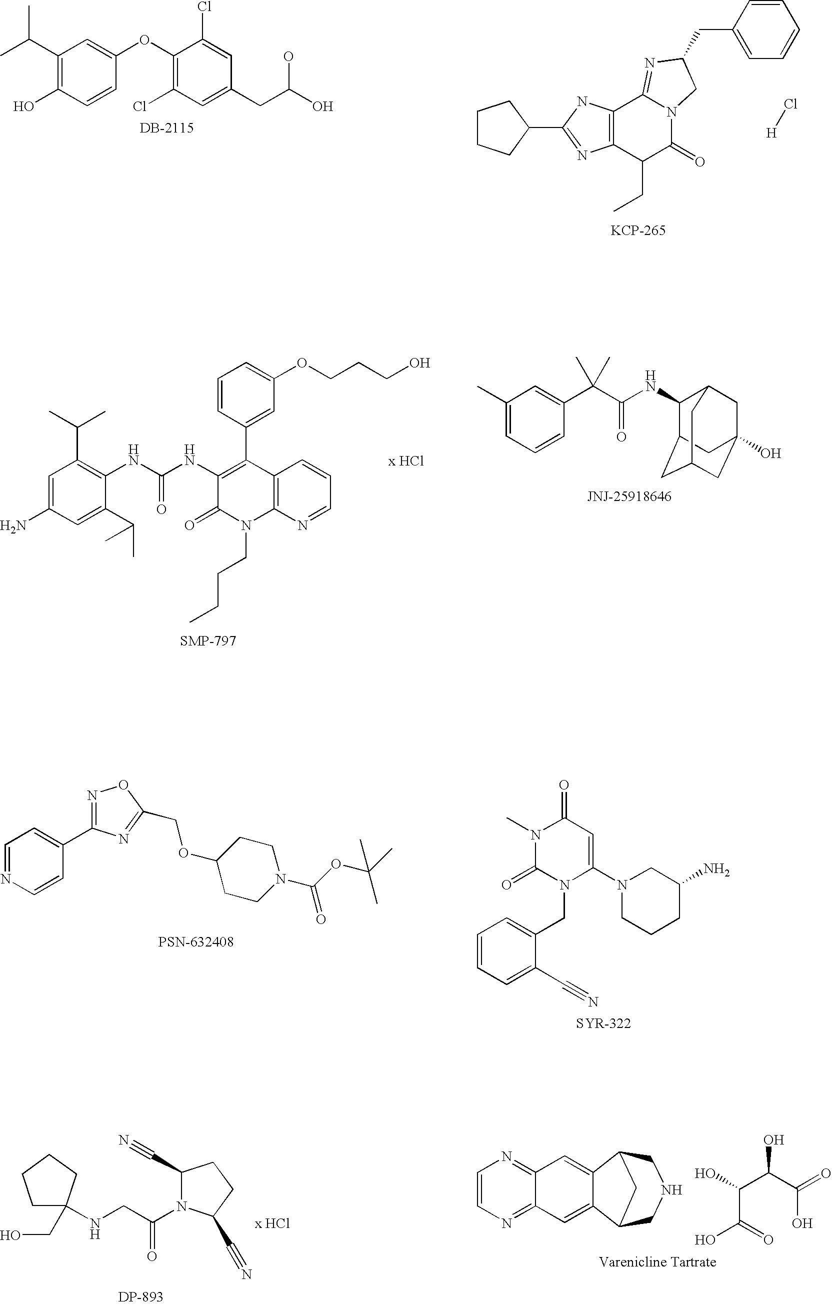

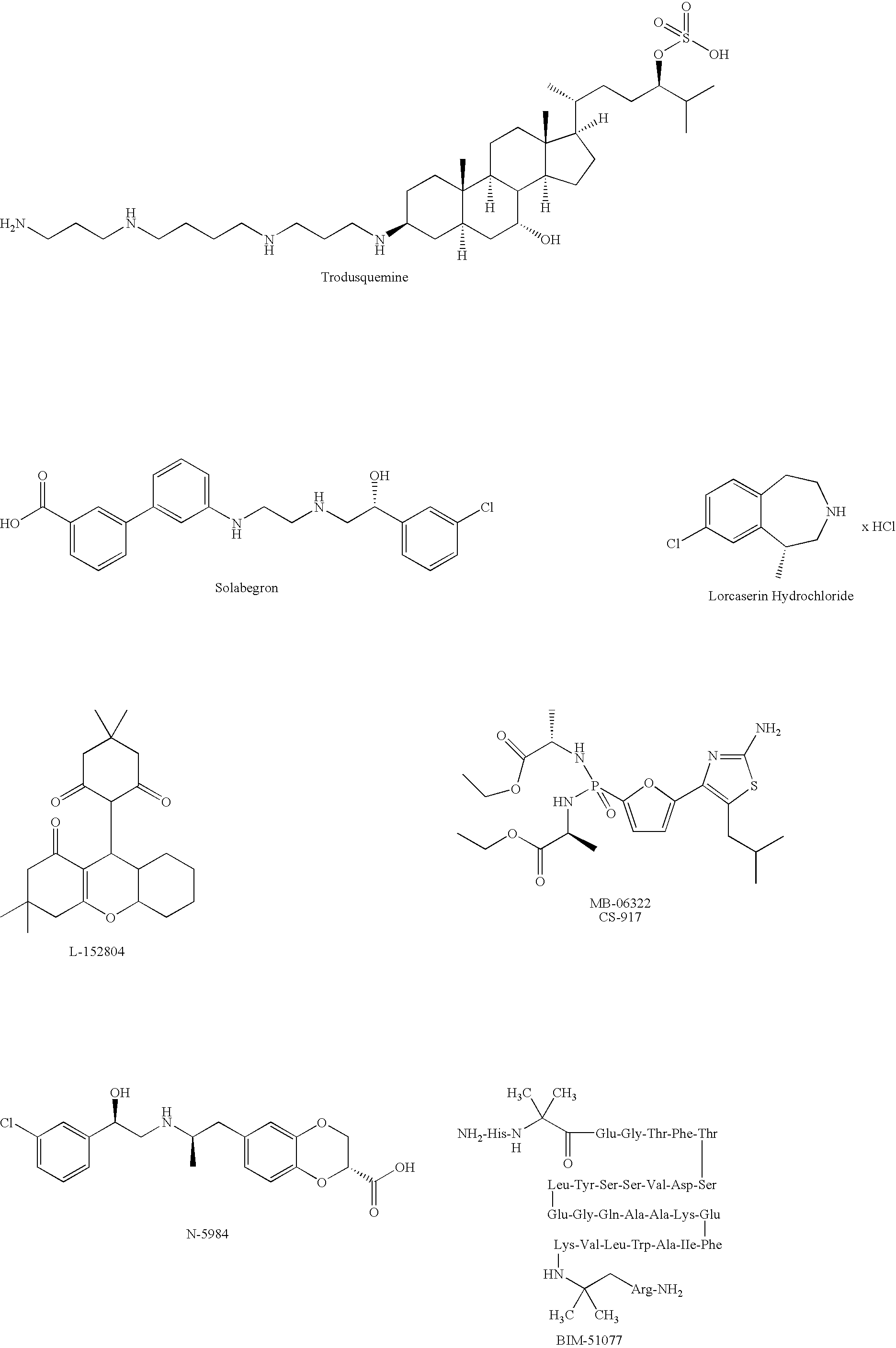

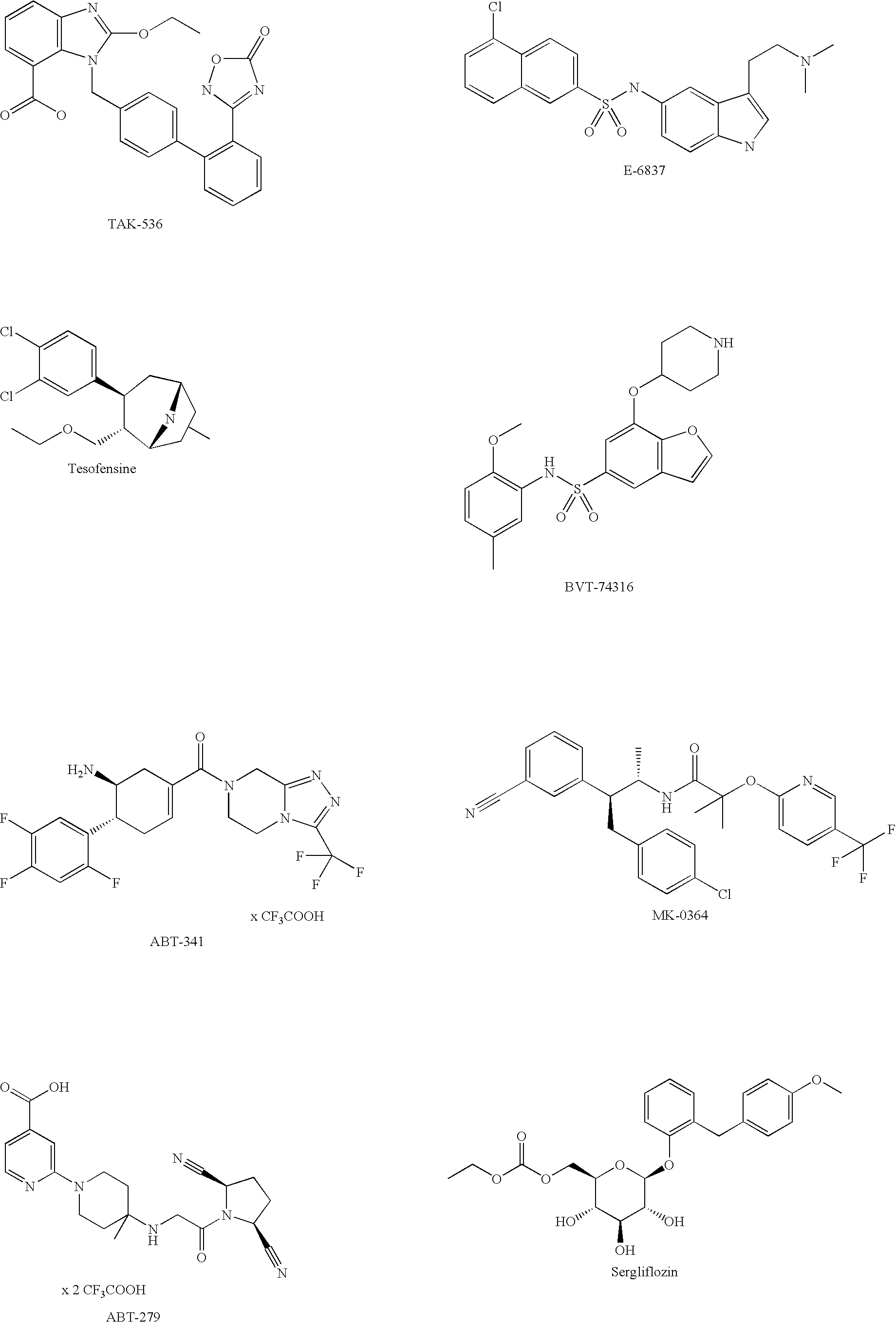

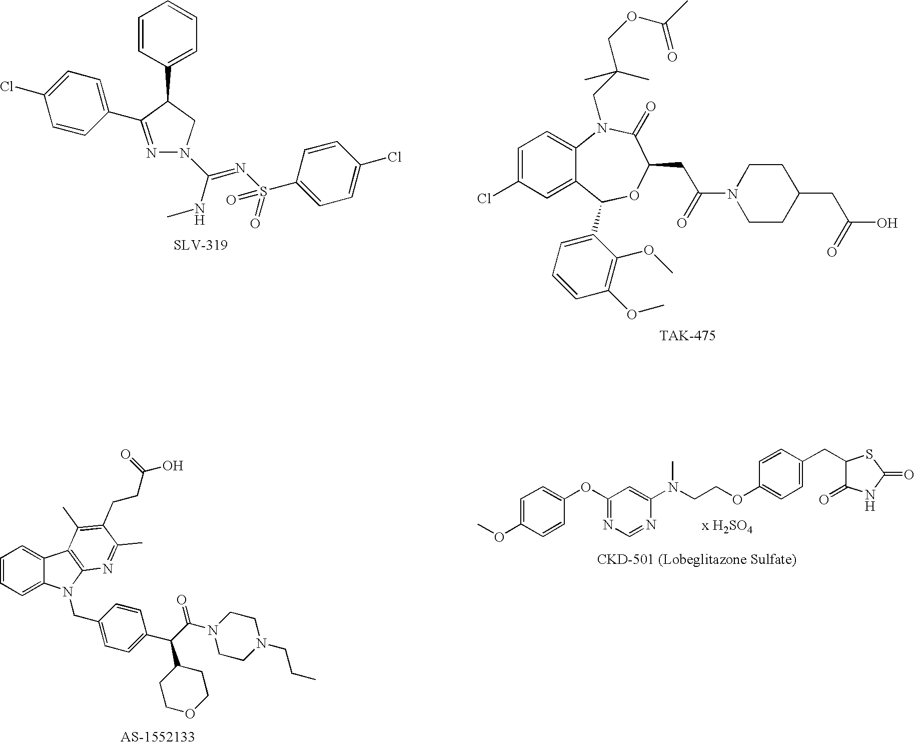

The following active ingredients are further suitable for combination products:

all antiepileptics mentioned in the Rote Liste 2006, Chapter 15;
all antihypertensives mentioned in the Rote Liste 2006, Chapter 17;
all hypotensives mentioned in the Rote Liste 2006, Chapter 19;
all anticoagulants mentioned in the Rote Liste 2006, Chapter 20;
all arteriosclerosis remedies mentioned in the Rote Liste 2006, Chapter 25;
all beta-receptor, calcium channel blockers and inhibitors of the renin-angiotensin system mentioned in the Rote Liste 2006, Chapter 27;
all diuretics and blood flow stimulators mentioned in the Rote Liste 2006, Chapters 36 and 37;
all anticraving drugs/agents for the treatment of addictive disorders mentioned in the Rote Liste 2006, Chapter 39;
all coronary agents and gastrointestinal drugs mentioned in the Rote Liste 2006, Chapters 55 and 60;
all migraine remedies, neuropathy products and antiParkinson agents mentioned in the Rote Liste 2006, Chapters 61, 66 and 70.

The invention further relates to methods for preparing the compound of the formula I and its salts as shown in scheme 1 and 2.

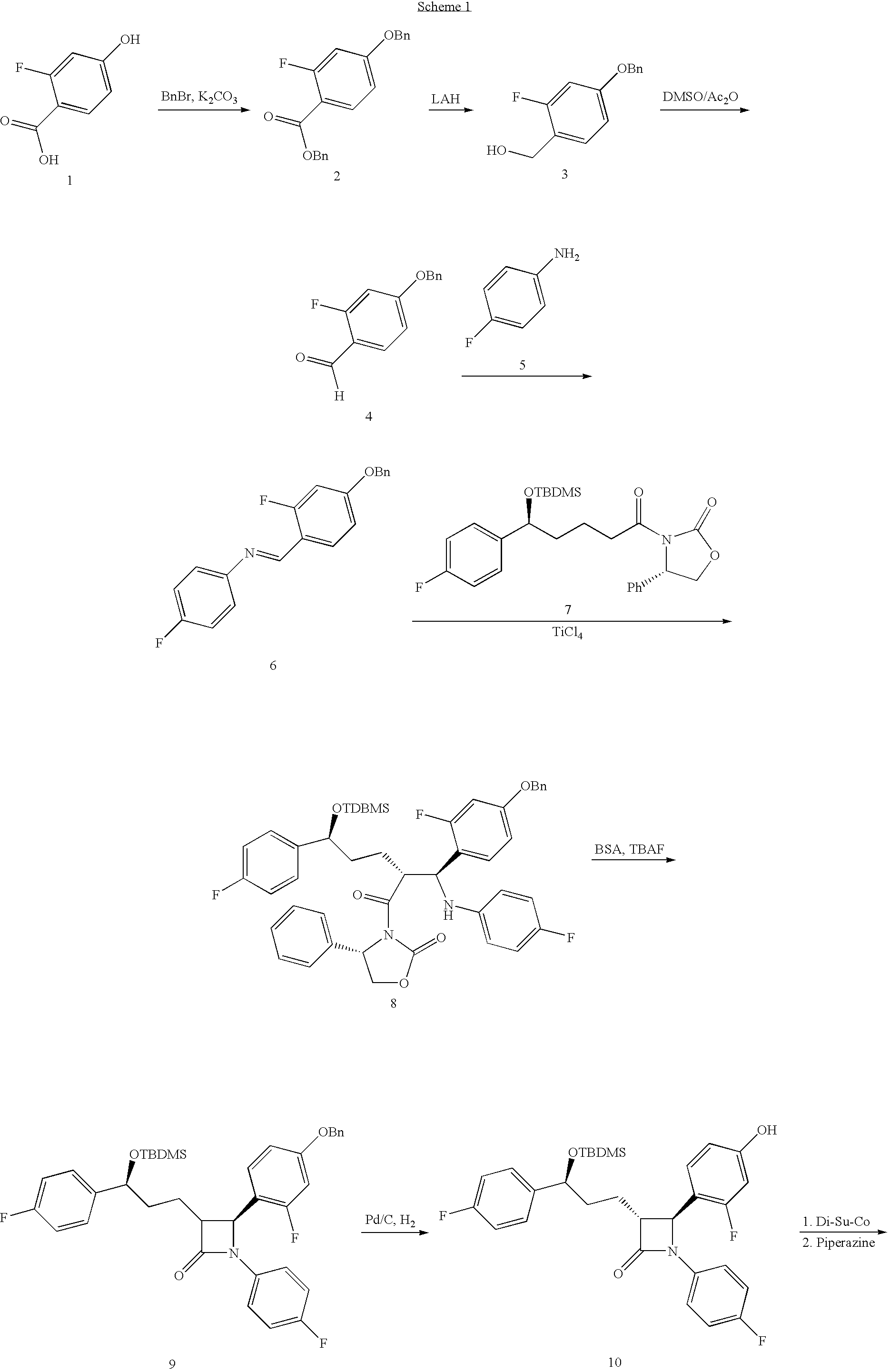

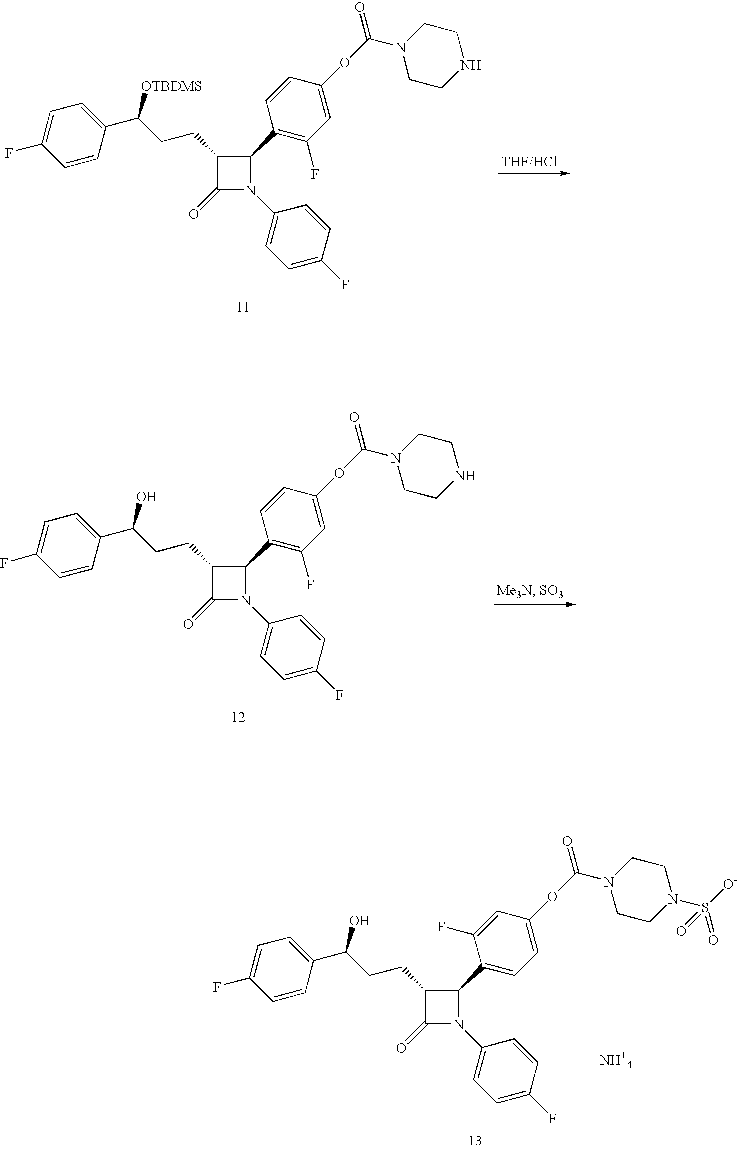

The intermediate 12 can also be synthesized by the following route:

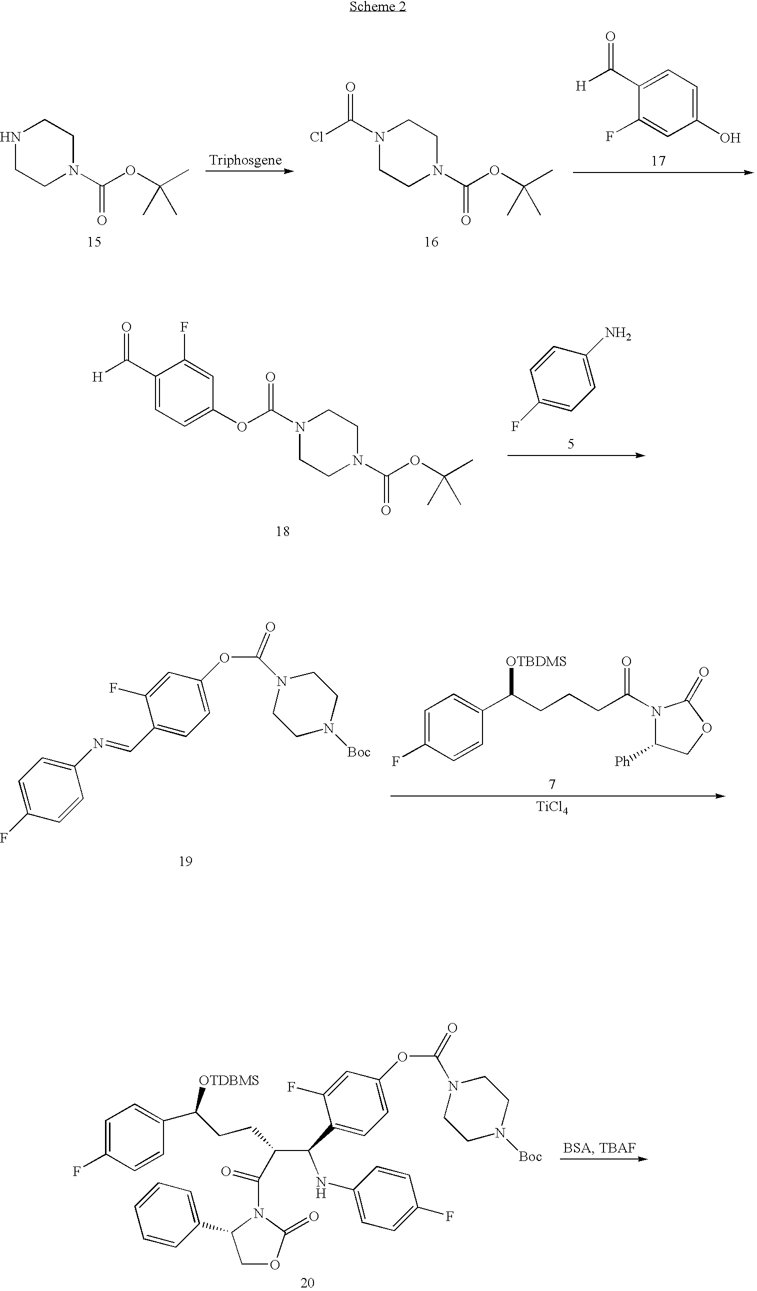

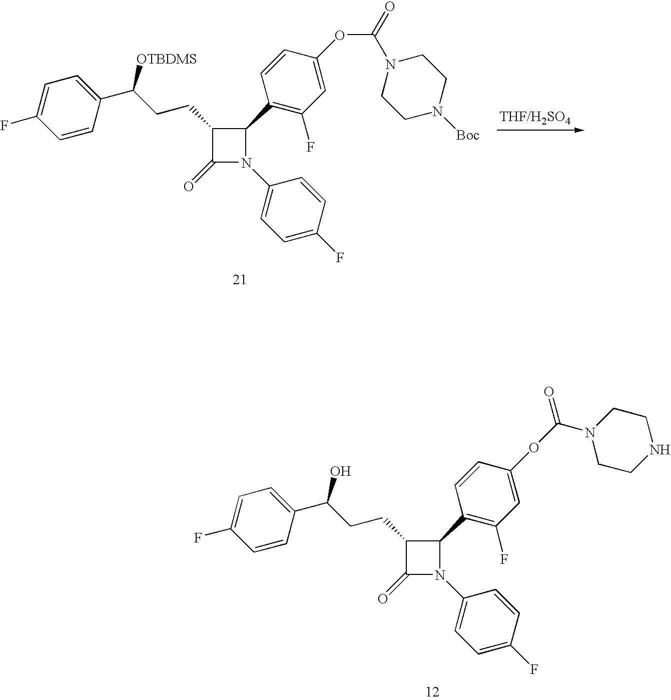

### Benzyl 4-benzyloxy-2-fluorobenzoate 2

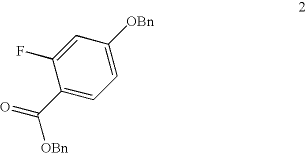

10.0 g (64 mmol) of 2-fluoro-4-hydroxybenzoic acid 1 (Aldrich) are suspended in 150 ml of DMF, 25 ml (200 mmol) of benzyl bromide and 40 g (290 mmol) of potassium carbonate. The reaction solution is left to stir at room temperature for 18 hour. For working up, 400 ml of n-heptane/ethyl:acetate (4:1) are added, and the mixture is extracted 3 times with water. The organic phase is filtered through silica gel and concentrated, and 22.5 g of perbenzylated crude product 2 are obtained.

### 4-Benzyloxy-2-fluorobenzyl alcohol 3

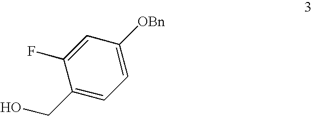

22.5 g (max 64 mmol) of crude product 2 are dissolved in 30 ml of tetrahydrofuran (THF), diluted with a further 300 ml of diethyl ether and cooled to 0° C. A 1 M lithium aluminum hydride solution in diethyl ether (80 ml) is slowly added dropwise at 0° C. and then stirred at 0° C. for 15 minutes. Excess lithium aluminum hydride is decomposed by adding 10 ml of ethyl acetate. In order to obtain a precipitate which can be filtered satisfactorily, 4 ml of water, 4 ml of 10% strength sodium hydroxide solution and 8 ml of water are cautiously added in succession. The precipitate is filtered through silica gel, washed with ethyl acetate and then concentrated. 19.8 g of crude product 3 are obtained.

### 4-Benzyloxy-2-fluorobenzaldehyde 4

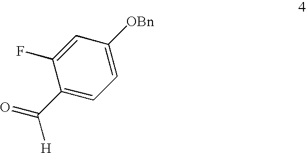

19.8 g of crude product 3 are dissolved in 200 ml of DMSO and 100 ml of acetic anhydride and left to stand at room temperature for 18 hours. This reaction solution is then diluted with 500 ml of n-heptane/ethyl acetate (2:1) and washed 3 times with saturated NaCl solution, filtered through silica gel and concentrated. Remaining acetic anhydride is evaporated off with toluene, and the residue is dissolved in a little n-heptane/ethyl acetate (2:1). 4.4 g of aldehyde 4 crystals are filtered off with suction. A further 1.6 g of crystals are obtained from the mother liquor after flash chromatography. Total yield 6 g (41% yield over 3 stages). Aldehyde 4 with molecular weight 230.24 (C₁₄H₁₁FO₂); MS (ESI⁺): 231.1 (M+H⁺).

### (4-Benzyloxy-2-fluorobenzylidene)(4-fluorophenyl)amine 6

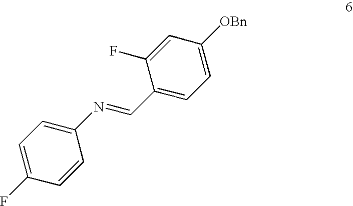

6.0 g (26.1 mmol) of aldehyde 4 and 5 ml (57 mmol) of p-fluoroaniline 5 (Fluka) are boiled with 250 ml of toluene with a water trap for 2 hours, and about 150 ml of toluene are distilled out during this. The remaining toluene is concentrated in a rotary evaporator, and the residue is purified by flash chromatography (n-heptane/ethyl acetate 2:1+1% triethylamine), and 8.34 g (98% yield) of imine 6 are obtained as a crystalline solid (from n-heptane/ethyl acetate).

### (S)-3-[(S)-2-[(4-Benzyloxy-2-fluorophenyl)(4-fluorophenylamino)methyl]-5-(tert-butyldimethylsilanyloxy)-5-(4-fluorophenyl)pentanoyl]-4-phenyloxazolidin-2-one 8

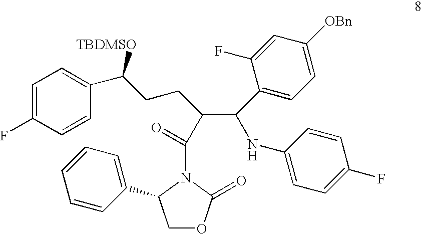

5.0 g (44.6 mmol) of oxazolidinone 7 are dissolved together with 9 ml of diisopropylethylamine in 120 ml of methylene chloride and cooled to 0° C. under argon. 38 ml of a 1M TiCl₄/methylene chloride solution are slowly added dropwise to this solution. It is then warmed to 20° C. for 5 minutes and subsequently cooled to −30° C. At −30° C., a solution of 8.3 g (25.7 mmol) of imine 6 in 100 ml of methylene chloride is added dropwise, and the mixture is stirred at −30° C. for 30 minutes. The reaction solution is extracted with 100 ml of water. The organic phase is filtered through 100 ml of silica gel. The aqueous phase is extracted once again with 80 ml of n-heptane/ethyl acetate (2:1), and the organic phase is used in order to wash the silica gel of the first filtration. The organic phase is concentrated, and 36 g of crude product 8 are obtained.

### 4-(4-Benzyloxy-2-fluorophenyl)-3-[(S)-3-(tert-butyldimethylsilanyloxy)-3-(4-fluorophenyl)propyl]-1-(4-fluorophenyl)azetidin-2-one 9

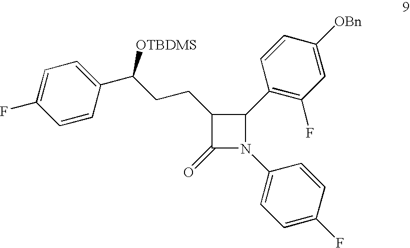

36 g of crude product 8 are dissolved in 500 ml of methyl tert-butyl ether (MTB ether). 40 ml of bistrimethylsilylacetamide (BSA) are added dropwise, and the mixture is cooled to 0° C. After addition of 20 ml of 1M tetrabutylammonium fluoride (TBAF) in THF, the mixture is allowed to warm to room temperature and is then stirred at room temperature for 1 hour. The reaction solution is filtered through silica gel and washed with ethyl acetate. The residue after removal of the solvent by distillation is purified by flash chromatography (n-heptane/ethyl acetate 4:1 to 2:1), and 12.3 g (74% over 2 stages as mixture of diastereomers) of beta-lactam 9 are obtained. Further reactions are carried out up to the sulfate 13 with the mixture of diastereomers. The crystalline ammonium salt of the sulfate 13 can then be separated by recrystallization to give the pure diastereomer.

### 3-[(S)-3-(tert-Butyldimethylsilanyloxy)-3-(4-fluorophenyl)propyl]-4-(2-fluoro-4-hydroxyphenyl)-1-(4-fluorophenyl)azetidin-2-one 10

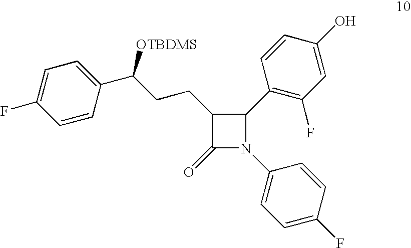

12.3 g (19.0 mmol) of lactam 9 are dissolved in 120 ml of methylene chloride and hydrogenated under 6 bar of hydrogen with 2.5 g of 10% Pd on activated carbon for 18 hours. The palladium/activated carbon is removed on a little silica gel and concentrated. 10.6 g of crude product 10 are obtained.

### 4-[3-[(S)-3-(tert-Butyldimethylsilanyloxy)-3-(4-fluorophenyl)propyl]-1-(4-fluorophenyl)-4-oxoazetidin-2-yl]-3-fluorophenyl piperazine-1-carbonate 11

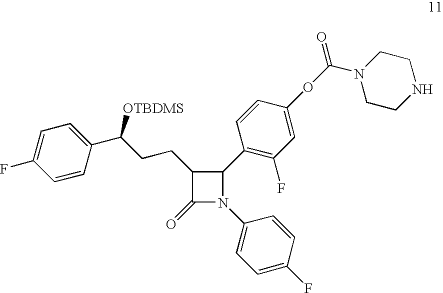

5.4 g (9.6 mmol) of compound 10 are dissolved in 50 ml of acetonitrile. 5 ml of triethylamine and 4 g (15.6 mmol) of Di-Su-CO (Fluka) are successively added, and the mixture is left to stand at room temperature for 90 minutes. The reaction solution is then added dropwise to a solution of 4 g of piperazine in 50 ml of acetonitrile, and the mixture is stirred for 3 hours. The heterogeneous reaction solution is purified directly by flash chromatography (methylene chloride/methanol/conc. ammonia 100/7/1, then 30/5/1, then 30/10/3), and 1.6 g of product 11 as colorless amorphous solid, and 3.35 g of precursor 10, are obtained.

### 3-Fluoro-4-{1-(4-fluorophenyl)-3-[(S)-3-(4-fluorophenyl)-3-hydroxypropyl]-4-oxoazetidin-2-yl}phenyl piperazine-1-carbonate 12

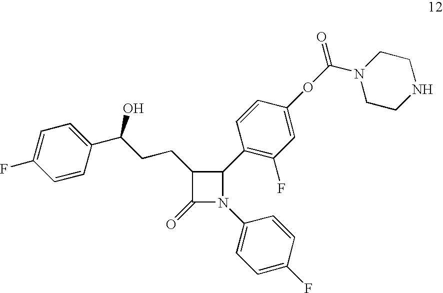

### A) According to Scheme 1:

1.6 g (2.5 mmol) of compound II are dissolved in 50 ml of THF. After addition of 15 ml of 2N aqueous HCl, the homogeneous solution is left to stand at room temperature for 16 hours. The solution is then basified by adding a methylene chloride/methanol/conc. ammonia (30/10/30) mixture and then concentrated. The residue is suspended in a little methylene chloride/methanol/conc. ammonia 30/5/1 and purified by flash chromatography (methylene chloride/methanol/conc. ammonia 30/5/1 then 30/10/3), and 1.11 g of compound 12 are obtained as an amorphous solid with molecular weight 539.56 (C₂₉H₂₈F₃N₃O₄); MS (ESI⁺): 522.28 (M+H⁺−H₂O).

### B) According to Scheme 2:

10 ml of 50% strength sulfuric acid are added dropwise to a solution of 5.2 g (6.90 mmol) of lactam 21 in 60 ml of tetrahydrofuran at 50° C. The solution is stirred at 50° C. for 2 hours, cooled to 5° C. and, at this temperature, basified with 70 ml of a dichloromethane/methanol/conc. ammonia (3/3/1) mixture. Filtration is followed by evaporation to dryness, and the product is purified by silica gel chromatography (dichloromethane/methanol/conc. ammonia 100/7/1 then 30/5/1). 2.52 g (68%) of the lactam 12 (diastereomerically pure; 3-fluoro-4-{(2S,3R)-1-(4-fluorophenyl)-3-[(S)-3-(4-fluorophenyl)-3-hydroxypropyl]-4-oxoazetidin-2-yl}phenyl piperazine-1-carbonate) are obtained as a viscous oil.

### tert-Butyl 3-fluoro-4-formylphenyl piperazine-1,4-dicarboxylate 18

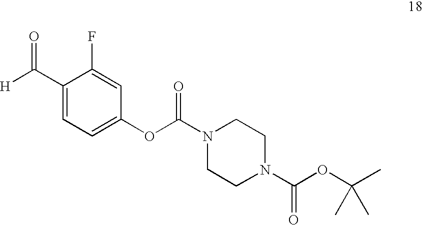

44 ml (0.55 mol) of pyridine are slowly added dropwise to a solution of 53.0 g (0.175 mol) of triphosgene in 500 ml of dichloromethane at 5° C., followed by a solution of 93.1 g (0.5 mol) of tert-butyl piperazine-1-carboxylate 15 (Fluka) in 280 ml of dichloromethane. The solution is stirred at 5° C. for 1 hour and at room temperature for 30 min and then 285 ml of 3N hydrochloric acid are added. After separation of the phases, the aqueous phase is extracted with dichloromethane and the combined organic phases are washed with water and sodium chloride solution. The solution is concentrated to a volume of 500 ml.

330 ml of N-methyl-2-pyrrolidone and 69.0 g (0.5 mol) of potassium carbonate are added to the resulting solution which contains the hydrolysis-sensitive acid chloride 16 (A. R. Gangloff, Bioorg. Med. Chem. Lett. 2000, 10, 2357). At 40° C., a solution of 61.0 g (0.435 mol) of 2-fluoro-4-hydroxybenzaldehyde 17 (Apollo Scientific) in 180 ml of N-methyl-2-pyrrolidone is added dropwise, and the suspension is then stirred at room temperature for 14 hours. Then, at 10° C., 500 ml of 2N hydrochloric acid are added dropwise, and the mixture mixed with 500 ml of ethyl acetate and 350 ml of water. After separation of the phases, the aqueous phase is extracted with ethyl acetate, and the combined organic phases are washed successively with saturated sodium bicarbonate solution and sodium chloride solution. The solution is concentrated to a volume of 200 ml and, at 50° C., 500 ml of n-heptane are added. The mixture is allowed to cool to room temperature, and the precipitated solid is filtered off. Drying results in 142 g (81%) of crystalline aldehyde 18 [C₁₇H₂₁FN₂O₅, M=352.37 g/mol]; MS (ESI⁺): 297.0 (M-tBu+2H).

### tert-Butyl 3-fluoro-4-{[(E)-4-fluorophenylimino]methyl}phenyl piperazine-1,4-dicarboxylate 19

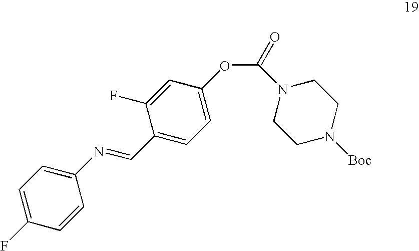

A suspension of 53.7 g (0.152 mol) of the aldehyde 18 in 150 ml of ethanol is mixed with 16.9 g (0.152 mol) of p-fluoroaniline 5 (Fluka) and refluxed for 3 hours. Then, at 65° C., 50 ml of diisopropyl ether are added dropwise, and the solution is cooled to room temperature. The precipitated solid is filtered off and dried. 61 g (90%) of crystalline imine 19 are obtained [C₂₃H₂₅F₂N₃O₄; ¹H NMR (d6-DMSO): δ (ppm)=8.7 (s, 1H), 8.1 (t, 1H), 7.4-7.1 (m, 6H), 3.6 (vs, 2H), 3.4 (bs, 6H), 1.4 (s, 9H)].

### tert-Butyl 4-[(1S,2R,5S)-5-(tert-butyldimethylsilanyloxy)-5-(4-fluorophenyl)-1-(4-fluorophenylamino)-2-((S)-4-phenyloxazolidin-2-one-3-carbonyl)pentyl]-3-fluorophenyl piperazine-1,4-dicarboxylate 20

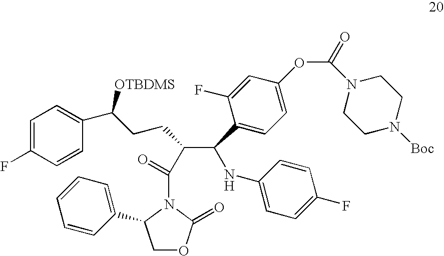

12 ml (69.6 mmol) of diisopropylethylamine and 32 ml (31.9 mmol) of a 1M titanium tetrachloride/dichloromethane solution are successively added dropwise to a solution of 13.7 g (29.1 mmol) of oxazolidinone 7 in 60 ml of dichloromethane at 0° C. The mixture is stirred at room temperature for 45 min and then cooled to −30° C. At this temperature, a solution of 14.2 g (31.9 mmol) of imine 19 in 35 ml of dichloromethane is added dropwise. The mixture is stirred at −30° C. for 2 hours and then a solution of 8 ml of acetic acid in 8 ml of dichloromethane is added dropwise. The reaction mixture is poured into 240 ml of 1N hydrochloric acid. After phase separation, the aqueous phase is extracted with dichloromethane, and the combined organic phases are washed successively with 5% strength sodium bicarbonate solution and water. After drying over sodium sulfate, most of the solvent is distilled off, and the remaining solution is mixed with 170 ml of ethanol and cooled to room temperature. The precipitated solid is filtered off with suction and recrystallized from ethanol. 14.1 g (53%) of diastereomerically pure crystalline product 20 are obtained [C₄₉H₅₉F₃N₄O₈Si, M=917.12 g/mol]; MS (ESI⁺): 918.4 (M+H).

### tert-Butyl 4-[(2S,3R)-3-[(S)-3-(tert-butyldimethylsilanyloxy)-3-(4-fluoro-phenyl)propyl]-1-(4-fluorophenyl)-4-oxoazetidin-2-yl]-3-fluorophenyl piperazine-14-dicarboxylate 21

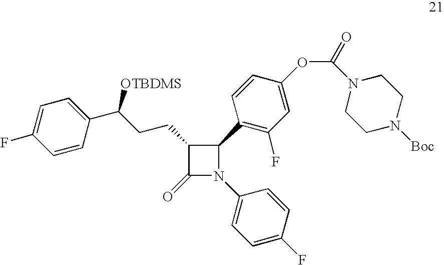

12 ml (45.9 mmol) of bistrimethylsilylacetamide are added to a solution of 14.0 g (15.3 mmol) of product 20 in 100 ml of toluene at room temperature, and the mixture is stirred for 30 min and then cooled to 0° C. At this temperature, 0.76 ml (0.8 mmol) of 1M tetrabutylammonium fluoride/tetrahydrofuran solution is added, and the mixture is stirred at room temperature for 2 hours. 40 ml of 1N hydrochloric acid are added to the reaction solution. After phase separation, the aqueous phase is extracted with toluene, and the combined organic phases are washed successively with 5% strength sodium bicarbonate solution and water. The solvent is distilled off and the residue is crystallized from diisopropyl ether/n-heptane. Drying results in 7.5 g (65%) of diastereomerically pure crystalline lactam 21 [C₄₀H₅₀F₃N₃O₆Si, M=753.94 g/mol]; MS (ESI⁺): 622.2 (M−OSiMe₂tBu).

### Ammonium 4-(3-fluoro-4-{(2S,3R)-1-(4-fluorophenyl)-3-[(S)-3-(4-fluoro-phenyl)-3-hydroxylpropyl]-4-oxoazetidin-2-yl}phenoxycarbonyl)piperazine-1-sulfonate 13

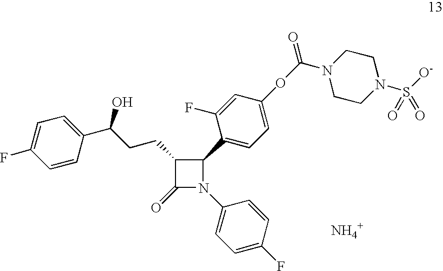

1.04 g (1.9 mmol) of compound 12 are dissolved in 20 ml of methanol and cooled to 0° C. Addition of 1 g (7.18 mmol) of trimethylamine-sulfotrioxide complex is followed by stirring at 0° C. for 2 hours. The reaction is mixed with 10 ml of methylene chloride/methanol/conc. ammonia 30/10/3, and the suspension is filtered through a little silica gel and washed with methylene chloride/methanol/conc. ammonia 30/10/3. The residue after concentration is purified by flash chromatography (methylene chloride/methanol/conc. ammonia 30/5/1, then 30/10/3, then 30/15/5). 1.2 g of sulfamide 13 are obtained. This is dissolved in a little methanol (2 to 3 ml) and then diluted with 30 ml of acetonitrile. This is followed by cautious evaporation in a rotary evaporator until crystallization starts (distill out about 15 ml). The crystals are filtered off with suction and washed with acetonitrile. 777 mg of crystalline product 13 are obtained (melting point 133-149° C.) with molecular weight 619.62 (C₂₉H₂₈F₃N₃O₇S×NH₃); MS (ESI⁺): 602.33 (M+H⁺−H₂O) and 355 mg of mother liquor. The crystalline product is diastereomerically pure and the mother liquor is a mixture of diastereomers.

### Sodium 4-(3-fluoro-4-{(2S,3R)-1-(4-fluorophenyl)-3-[(S)-3-(4-fluorophenyl)-3-hydroxypropyl]-4-oxoazetidin-2-yl}phenoxycarbonyl)piperazine-1-sulfonate 14

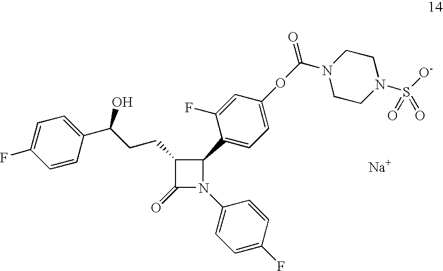

100 mg of the compound 13 are dissolved in a mixture of 3 ml of acetonitrile and 3 ml of water, and an excess of sodium bicarbonate is added. The mixture is stirred at room temperature for one hour and concentrated in a rotary evaporator. The residue is taken up in methanol/water and again concentrated. This procedure is repeated a few times. Crystalline sodium salt 14 is obtained as hydrate with a melting point of 175° C.

Alternatively, the sodium salt as well as the potassium, calcium, magnesium, zinc, L-lysine, L-arginine, tris(hydroxymethyl)aminomethane and N-methyl-D-glucamine salts can be obtained by ion exchange chromatography.

The compound of the invention of the formula I (ammonium salt) was tested for its effect using the method described below:

NMRI mice (in groups of n=4-6) are kept with a standard diet (altromin, lage (lippe)) in metabolism cages. The animals are fasted from the afternoon before administration of the radioactive tracer (¹⁴C-cholesterol) and adapted to wire grids.

In addition, the animals are labeled with ³H-TCA (taurocholic acid) s.c. 24 hours before the oral administration of the test meal (¹⁴C-cholesterol in Intralipid® 20, Pharmacia-Upjohn) (e.g. 1 μCi/mouse to 5 μCi/rat).

Cholesterol absorption test: 0.25 ml/mouse Intralipid® 20 (Pharmacia-Upjohn) (spiking with 0.25 μCi ¹⁴C-cholesterol in 0.1 mg of cholesterol) is administered orally by gavage.

Test substances are made up separately in 0.5%/(methylcellulose (Sigma)/5% Solutol (BASF, Ludwigshafen) or a suitable vehicle.

The volume for administration of the test substance is 0.5 ml/mouse. The test substance is administered immediately before the test meal (Intralipid with ¹⁴C-cholesterol label) (cholesterol absorption test).

The livers are removed, homogenized and combusted in aliquots in an oximate (model 307, Packard) to determine the amount of ¹⁴C-cholesterol taken up/absorbed.

### Evaluation:

### Liver Samples:

The amount of ¹⁴C-cholesterol taken up in the liver is related to the dose administered. The ED₅₀ values are interpolated from a dose-effect plot as the dose which halves the uptake of ¹⁴C-cholesterol in the liver (50%) relative to a control group.

The following ED₅₀ demonstrates the activity of the compound of the invention of the formula I

| Example No. | ED₅₀ (liver) [mg/mouse] |
|---|---|
| I (ammonium salt) | 0.01 |

It is evident from the table that the compound of the formula I (ammonium salt) has a very good cholesterol-lowering effect.

The compound of most similar structure from WO 2004/000804 was selected as comparison compound, being example LVIII disclosed therein.

| Example No. | ED₅₀ (liver) [mg/mouse] |
|---|---|
| LVIII from WO 2004/000804 | 0.1 |

The compound of the invention of the formula I thus has an activity which is 10 times better than the comparison compound LVIII from WO 2004/000804.

We claim:

1. A compound of formula I 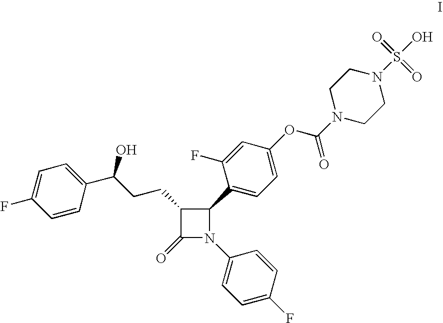
or a pharmaceutically acceptable salt thereof.

2. A pharmaceutical composition comprising the compound according to claim 1 or a pharmaceutically acceptable salt thereof, in combination with at least one pharmaceutically acceptable carrier or excipient.

3. The pharmaceutical composition according to claim 2, further comprising at least one additional active ingredient.

4. The pharmaceutical composition according to claim 3, wherein the additional active ingredient is a compound that normalize lipid metabolism.

5. The pharmaceutical composition according to claim 3, wherein the additional active ingredient is selected from the group consisting of antidiabetics, hypoglycemic active ingredients, antiobesity agents, anorectics, HMGCOA reductase inhibitors, cholesterol absorption inhibitors, PPAR gamma agonists, PPAR alpha agonists, PPAR alpha agonists, PPAR gamma agonists, PPAR delta agonists, partial PPAR gamma agonists, partial PPAR gamma antagonists, fibrates, MTP inhibitors, CETP inhibitors, bile acid absorption inhibitors, polymeric bile acid adsorbents, LDL receptor inducers, ACAT inhibitors, antioxidants, vitamins, lipoprotein lipase modulators, ATP-citrate lyase inhibitors, squalene synthetase inhibitors, lipoprotein(a) antagonists, lipase inhibitors, insulins, GLP-1 derivatives, GLP-1, sulfonylureas, biguanides, meglitinides, thiazolidinediones, α-glucosidase inhibitors, active ingredients which act on the ATP-dependent potassium channel of the beta cells, glycogen phosphorylase inhibitors, glucagon receptor antagonists, activators of glucokinase, inhibitors of gluconeogenesis, inhibitors of fructose-1,6-bisphosphatase, modulators of glucose transporter 4 inhibitors of glutamine-fructose-6-phosphate amidotransferase, inhibitors of dipeptidylpeptidase IV, inhibitors of 11-beta-hydroxysteroid dehydrogenase 1, inhibitors of protein tyrosine phosphatase 1B, modulators of the sodium-dependent glucose transporter 1, modulators of the sodium-dependent glucose transporter 2, GPR40 modulators, inhibitors of hormone-sensitive lipase, inhibitors of acetyl-CoA carboxylase, inhibitors of phosphoenolpyruvate carboxykinase, inhibitors of glycogen synthase kinase-3, CART modulators, NPY antagonists, peptide YY 3-36, cannabinoid receptor 1 antagonist, MCH receptor antagonists, MC4 agonists, orexin antagonists, histamine H3 agonists, CRF antagonists, CRF BP antagonists, urocortin agonists, β3 agonists, melanocyte-stimulating hormone agonists, CCK-A agonists, serotonin reuptake inhibitors, mixed serotoninergic and noradrenergic compounds, 5HT receptor agonists, 5-HT2C receptor agonists, 5-HT6 receptor antagonists, bombesin agonists, galanin antagonists, human growth hormone, AOD-9604, growth hormone-releasing compounds, ghrelin antagonists, TRH agonists, uncoupling protein 2 modulators, uncoupling protein 3 modulators, leptin, leptin agonists, DA agonists, lipase/amylase inhibitors, RXR modulators, inhibitors of diacylglycerol O-acyltransferase, inhibitors of fatty acid synthase, oxyntomodulin, oleoyl-estrone, agonists of the thyroid hormone receptor, TR-β agonists and amphetamines.

6. A method for treating a lipid metabolism disorder, hyperlipidemia, arteriosclerotic manifestation, or insulin resistance, or for lowering the serum cholesterol level, in a patient in need thereof, comprising administering to the patient a pharmaceutically effective amount of the compound according to claim 1 or a pharmaceutically acceptable salt thereof.

7. A method for manufacturing a pharmaceutical composition comprising the compound according to claim 1 or a pharmaceutically acceptable salt thereof, which comprises mixing the compound according to claim 1 or the pharmaceutically acceptable salt thereof with a pharmaceutically acceptable carrier or excipient, and converting this mixture into a form suitable for administration.

8. A compound of formula 12

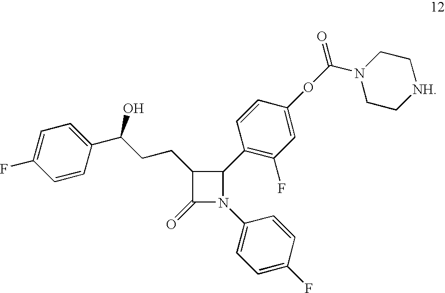

9. A compound of formula 18

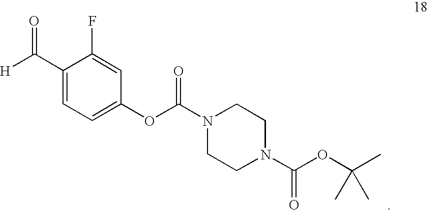

10. A compound of formula 19

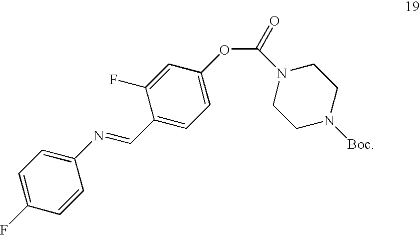

11. A compound of formula 20

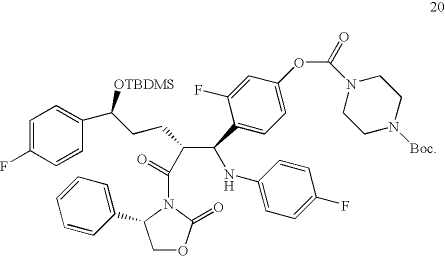

12. A compound of formula 21 [chemical structure]
